(19) United States
(12) Patent Application Publication
(10) Pub. No.: US 2012/0238841 A1
(43) Pub. Date: Sep. 20, 2012

(54) Sample capture in one step for test strips

(76) Inventors: Mark Castle, Pleasanton, CA (US); Jeffrey Perry, Sunnyvale, CA (US); Thomas Shoup, Los Altos, CA (US); Rodney Kugizaki, Tuscon, AZ (US)

(21) Appl. No.: 13/086,453

(22) Filed: Apr. 14, 2011

Related U.S. Application Data
(63) Continuation-in-part of application No. 12/744,514, filed on May 25, 2010.
(60) Provisional application No. 61/324,659, filed on Apr. 15, 2010.

(51) Int. Cl. A61B 5/151; A61B 5/1455; A61B 5/145; A61B 5/1468

(52) U.S. Cl. 600/316; 600/347; 600/365

(57) ABSTRACT

A test strip is provided with sample capture that provides for a one step process to achieve a lancing event, sample capture and sample transport in a sensor design that supports one step testing. In various embodiments, the present invention provides for one step testing by, (i) analyte sample capture layout; (ii) analyte sample capture and transport configurations; (iii) structures of sample capture; (iv) processes for forming sample transport, and the like.

### BACKGROUND OF THE INVENTION

1. Field of the Invention

The present invention relates generally to the collection of body fluid and more specifically, the use of sample capture with a test strip to provide one step to obtain body fluid and analyte measurement.

2. Description of Related Art

The treatment of diabetes requires frequent monitoring of levels of blood glucose. This is traditionally done in a series of steps involving the preparation of a lancing device, preparation of a glucose meter, lancing a finger, transporting the resulting blood drop to the meter, and finally obtaining a blood glucose reading.

Lancing devices are known in the medical health-care products industry for piercing the skin to produce blood for analysis. Biochemical analysis of blood samples is a diagnostic tool for determining clinical information. Many point-of-care tests are performed using capillary whole blood, the most common being monitoring diabetic blood glucose level. Other uses for this method include the analysis of oxygen and coagulation based on Prothrombin time measurement. Typically, a drop of blood for this type of analysis is obtained by making a small incision in the fingertip, creating a small wound, which generates a small blood droplet on the surface of the skin.

Early methods of lancing included piercing or slicing the skin with a needle or razor. Current methods utilize lancing devices that contain a multitude of spring, cam and mass actuators to drive the penetrating member. These include cantilever springs, diaphragms, coil springs, as well as gravity plumbs used to drive the penetrating member. Typically, the device is pre-cocked or the user cocks the device. The device is held against the skin and mechanically triggers the ballistic launch of the penetrating member. The forward movement and depth of skin penetration of the penetrating member is determined by a mechanical stop and/or dampening, as well as a spring or cam to retract the penetrating member. Spontaneous blood droplet generation is dependent on reaching the blood capillaries and venuoles, which yield the blood sample.

As lancing devices have become more advanced, so they have become more complex, using lower and lower volumes of blood or body fluid. There may be difficulty transferring low volumes of fluid from tissue to the device.

### SUMMARY

An object of the present invention is to provide a fully integrated, one step glucose diagnostic system, and its method of manufacture, where the user can place its finger on the device, press a button and get an accurate glucose reading.

Another object of the present invention is to provide a fully integrated, one step glucose diagnostic system, and its method of manufacture, that has seamless, automatic series of steps to lance the user's finger, draw blood, capture and transport the blood to a sensor and report a result.

Yet another object of the present invention is to provide a fully integrated, one step glucose diagnostic system, and its method of manufacture, for one step glucose measurement using sample capture, sample transport and measurement with an electrochemical sensor.

A further object of the present invention is to provide a fully integrated, one step glucose diagnostic system, and its method of manufacture, for one step glucose measurement that has structures for allowing a lancing event to be conducted, collecting a sample, transporting a sample and measuring the sample.

Another object of the present invention is to provide a fully integrated, one step glucose diagnostic system, and its method of manufacture, for one step glucose measurement that has structures for allowing a lancing event to be conducted, collecting a sample, transporting a sample and measuring the sample, where the structures are closely fluidicly coupled, such that a sample, expressed from a lancing event, presents itself at a prescribed location, and the structures enable the collection of this sample and it is subsequently transported to the measurement cell.

Yet another object of the present invention is to provide a glucose diagnostic system, and its method of manufacture, with a glucose sensor with structures that enables a lancing event, accomplish the sample capture and sample transport functions in a sensor design in one step testing.

Still another object of the present invention is to provide a glucose diagnostic system, and its method of manufacture, where a capillary flow is provided for blood to travel directly from a wound to the sensor port on a housing, and thus the volume of blood produced at the wound site, regardless of its droplet geometry, is completely transported to the analyte detecting member.

These and other objects of the present invention are achieved in a test strip device that has a first substrate with a first electrode and a second substrate with a second electrode. The second substrate includes a fluid passage way between the first and second substrates. A spacer layer includes an aperture coupled to the fluid passage way and positioned between the first and second electrodes. A reaction zone/sensor is formed between the first and second electrodes. A hydrophilic sample collection structure is provided.

In another embodiment, a test strip device for testing a biologic analyte obtained by lancing a finger includes an aperture in the test strip providing a path for a penetrating member. A sample-capture feature and a sample-collection feature are provided. A transport pathway moves the analyte to a specified portion of the test strip for reaction with a reagent and measurement of the reaction products.

In another embodiment, a test strip device has an aperture in a test strip that provides a path for a penetrating member. A sample-capture feature and a sample-collection feature are included. A transport pathway is created by covering the substrate of the test strip with a cover layer which provides a two-dimensional capillary area over which the analyte spreads automatically by means of capillary forces and in which reagent exists within said capillary area which reacts with the analyte such that the optical properties of the two-dimensional capillary area are changed in proportion to the concentration of the analyte and measurement of said concentration is by optical reflectance, transmission, or fluorescence.

In anther embodiment, a test strip device includes an aperture in the test strip to provide a path for a penetrating member. Sample-capture and sample-collection features are included in which the sample-collection feature is at least one of, a micro-fluidic hydrophilic structure containing reagent which reacts with an analyte.

### BRIEF DESCRIPTION OF THE DRAWINGS

FIG. 1 illustrates an embodiment of a controllable force driver in the form of a cylindrical electric penetrating member driver using a coiled solenoid-type configuration.

FIG. 2A illustrates a displacement over time profile of a penetrating member driven by a harmonic spring/mass system.

FIG. 2B illustrates the velocity over time profile of a penetrating member driver by a harmonic spring/mass system.

FIG. 2C illustrates a displacement over time profile of an embodiment of a controllable force driver.

FIG. 2D illustrates a velocity over time profile of an embodiment of a controllable force driver.

FIG. 3 is a diagrammatic view illustrating a controlled feed-back loop.

FIG. 4 is a perspective view of a tissue penetration device having features of the invention.

FIG. 5 is an elevation view in partial longitudinal section of the tissue penetration device of FIG. 4.

FIG. 6A shows one embodiment of a device which may use the present invention.

FIG. 6B shows one embodiment of a cartridge according to the present invention.

FIG. 7 is a perspective view of one embodiment with mesh on a cartridge.

FIG. 8 is a view showing a penetrating member diameter.

FIG. 9 shows one embodiment of the invention with a mesh with an opening for penetrating member exit.

FIGS. 10A through 10C show various embodiments of sample capture devices.

FIG. 11 is a side view of a sample capture device.

FIGS. 12A through 12D show various embodiments of sample capture devices.

FIG. 13 shows one method of manufacturing a sample capture device.

FIGS. 14 through 16 show other configurations of a device according to the present invention.

FIG. 17 shows one method of manufacturing a sample capture device.

FIG. 18 through 21 show configurations of sample capture devices.

FIGS. 22(a) and 22(b), an analyte diagnostic system is provided that uses one or more test strips with sample capture

FIGS. 23 and 24 are exploded views of a test strip of FIGS. 22(a) and 22(b).

FIG. 25 illustrates one embodiment of a test strip with sample capture positioned adjacent to a sensor/reaction zone, but does not impinge on the sensor/reaction zone, to provide a close fluidic coupling.

FIG. 26 illustrates an embodiment of a strip with a penetrating member axis that is perpendicular to a plane of the test strip.

FIGS. 26(a) through 26(j) illustrates various process flow steps in creating the FIG. 26 embodiment.

FIG. 27 illustrates another embodiment of a strip with sample capture for a one step bleed to read.

FIGS. 27(a) through 27(i) illustrates various process flow steps in creating the FIG. 27 embodiment.

FIG. 28 illustrates an embodiment of a strip with sample capture provided through a top of a sensor/reaction zone.

FIGS. 28(a) through 28(j) illustrates various process flow steps in creating the FIG. 28 embodiment.

FIG. 29 illustrates an embodiment of a strip with sample capture that has a lancing aperture in a substrate for a needle to pass through.

FIGS. 29(a) through 29(h) illustrates various process flow steps in creating the FIG. 29 embodiment.

FIG. 30 illustrates an embodiment of a strip with sample capture placed on the edge of the sensor/reaction zone channel, and impinges into the sensor/reaction zone.

FIGS. 30(a) through 30(h) illustrates various process flow steps in creating the FIG. 30 embodiment.

FIG. 31 illustrates an embodiment of a strip with a sample capture structure orthogonal to a plane of the strip.

FIGS. 31(a) through 31(l) illustrates various process flow steps in creating the FIG. 31 embodiment.

FIG. 32 illustrates an embodiment of the test strip that integrates the following structure and capabilities in an effective way to, (i) generate a sample is through using a controlled lancing event, where the profile of the lancing event is controlled; (ii) collect a blood sample and have the lancing event occur such that a lancing needle path is perpendicular to the plane of a circular sample collection structure; and (iii) transport the sample, once collected, through a hydrophilic treated capillary connecting the sample collection to the sensor.

FIG. 33 illustrates different sensors of the FIG. 32 embodiment.

FIGS. 33(a) through 33(f) illustrates an embodiment of process flow steps for manufacture of the FIGS. 32 and 33 strip.

FIGS. 34 through 36 are views of the strip 600.

### DESCRIPTION OF THE SPECIFIC EMBODIMENTS

It is to be understood that both the foregoing general description and the following detailed description are exemplary and explanatory only and are not restrictive of the invention, as claimed. It may be noted that, as used in the specification and the appended claims, the singular forms “a”, “an” and “the” include plural referents unless the context clearly dictates otherwise. Thus, for example, reference to “a material” may include mixtures of materials, reference to “a chamber” may include multiple chambers, and the like. References cited herein are hereby incorporated by reference in their entirety, except to the extent that they conflict with teachings explicitly set forth in this specification.

In this specification and in the claims which follow, reference will be made to a number of terms which shall be defined to have the following meanings: “Optional” or “optionally” means that the subsequently described circumstance may or may not occur, so that the description includes instances where the circumstance occurs and instances where it does not. For example, if a device optionally contains a feature for analyzing a blood sample, this means that the analysis feature may or may not be present, and, thus, the description includes structures wherein a device possesses the analysis feature and structures wherein the analysis feature is not present.

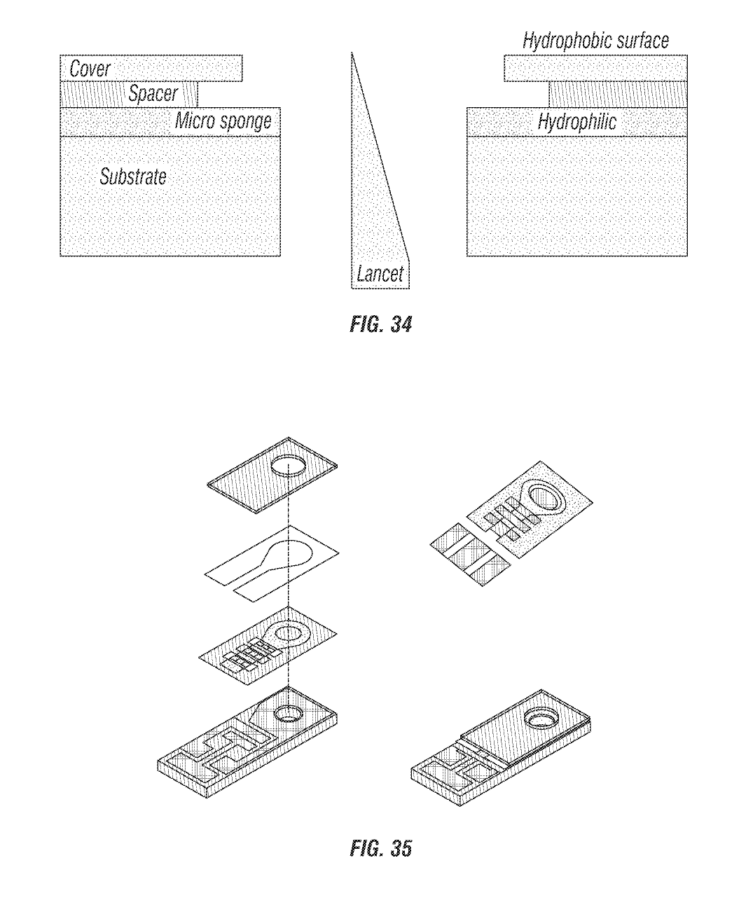

FIGS. 34 through 36 illustrate an embodiment of a strip of the present invention with, (i) a penetrating member path through the strip; (ii) sample capture feature with cover that has hole larger than the micro sponge with a hydrophobic on the upper surface; (iii) and a sample collection feature, where the hydrophilic micro sponge can surround the penetrating member and exposed to the skin on a finger when in close proximity; and spacer forms the walls of the sample transport feature.

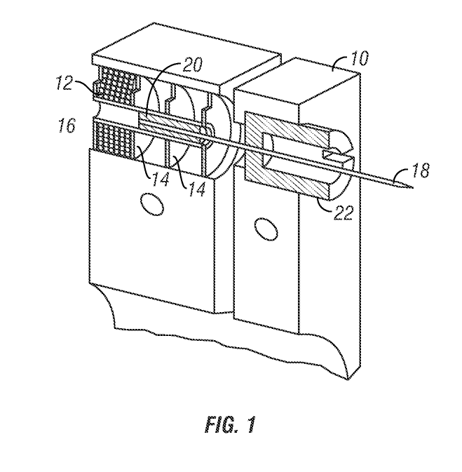

The present invention may be used with a variety of different penetrating member drivers. It is contemplated that these penetrating member drivers may be spring based, solenoid based, magnetic driver based, nanomuscle based, or based on any other mechanism useful in moving a penetrating member along a path into tissue. It should be noted that the present invention is not limited by the type of driver used with the penetrating member feed mechanism. One suitable penetrating member driver for use with the present invention is shown in FIG. 1.

This is an embodiment of a solenoid type electromagnetic driver that is capable of driving an iron core or slug mounted to the penetrating member assembly using a direct current (DC) power supply. The electromagnetic driver includes a driver coil pack that is divided into three separate coils along the path of the penetrating member, two end coils and a middle coil. Direct current is alternated to the coils to advance and retract the penetrating member. Although the driver coil pack is shown with three coils, any suitable number of coils may be used, for example, 4, 5, 6, 7 or more coils may be used.

Referring to the embodiment of FIG. 1, the stationary iron housing 10 may contain the driver coil pack with a first coil 12 flanked by iron spacers 14 which concentrate the magnetic flux at the inner diameter creating magnetic poles. The inner insulating housing 16 isolates the penetrating member 18 and iron core 20 from the coils and provides a smooth, low friction guide surface. The penetrating member guide 22 further centers the penetrating member 18 and iron core 20. The penetrating member 18 is protracted and retracted by alternating the current between the first coil 12, the middle coil, and the third coil to attract the iron core 20. Reversing the coil sequence and attracting the core and penetrating member back into the housing retracts the penetrating member. The penetrating member guide 22 also serves as a stop for the iron core 20 mounted to the penetrating member 18.

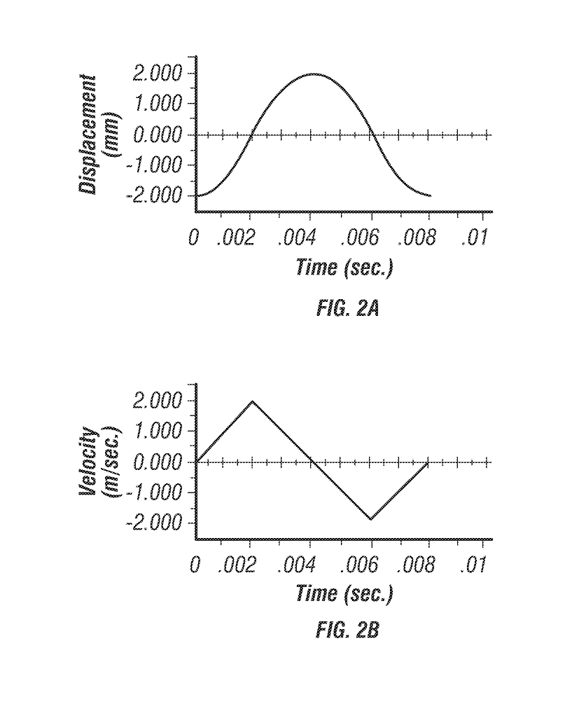

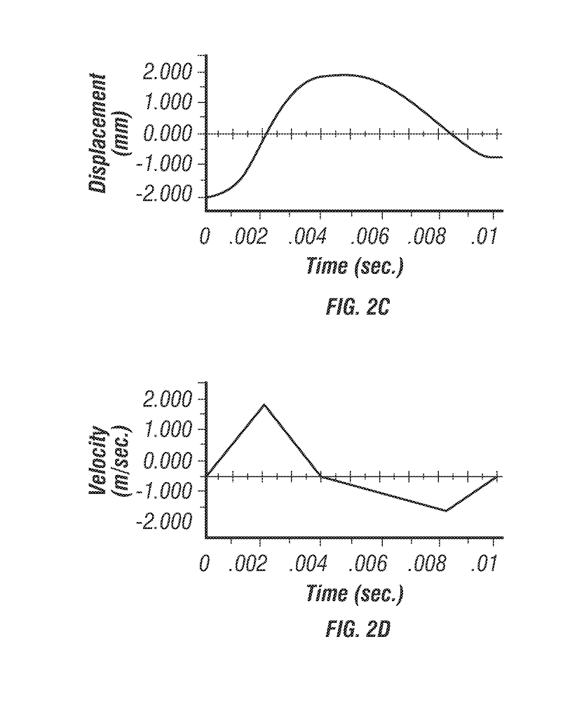

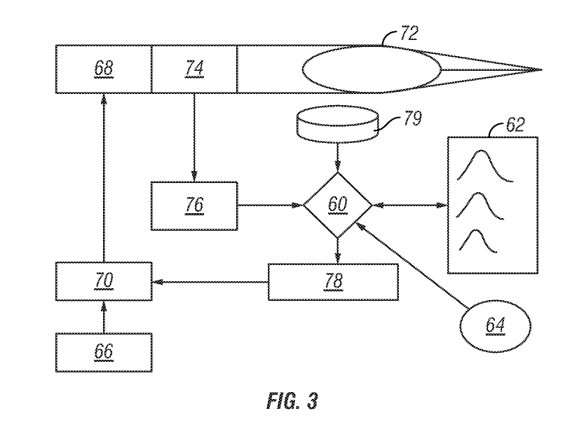

As discussed above, tissue penetration devices which employ spring or cam driving methods have a symmetrical or nearly symmetrical actuation displacement and velocity profiles on the advancement and retraction of the penetrating member as shown in FIGS. 2 and 3. In most of the available penetrating member devices, once the launch is initiated, the stored energy determines the velocity profile until the energy is dissipated.

Controlling impact, retraction velocity, and dwell time of the penetrating member within the tissue can be useful in order to achieve a high success rate while accommodating variations in skin properties and minimize pain. Advantages can be achieved by taking into account of the fact that tissue dwell time is related to the amount of skin deformation as the penetrating member tries to puncture the surface of the skin and variance in skin deformation from patient to patient based on skin hydration.

In this embodiment, the ability to control velocity and depth of penetration may be achieved by use of a controllable force driver where feedback is an integral part of driver control. Such drivers can control either metal or polymeric penetrating members or any other type of tissue penetration element. The dynamic control of such a driver is illustrated in FIG. 2C which illustrates an embodiment of a controlled displacement profile and FIG. 2D which illustrates an embodiment of a the controlled velocity profile. These are compared to FIGS. 2A and 2B, which illustrate embodiments of displacement and velocity profiles, respectively, of a harmonic spring/mass powered driver. Reduced pain can be achieved by using impact velocities of greater than about 2 m/s entry of a tissue penetrating element, such as a penetrating member, into tissue.

Other suitable embodiments of the penetrating member driver are described in commonly assigned, copending U.S. patent application Ser. No. 10/127,395, (Attorney Docket No. 38187-2551) filed Apr. 19, 2002 and previously incorporated herein.

FIG. 3 illustrates the operation of a feedback loop using a processor 60. The processor 60 stores profiles 62 in non-volatile memory. A user inputs information 64 about the desired circumstances or parameters for a lancing event. The processor 60 selects a driver profile 62 from a set of alternative driver profiles that have been preprogrammed in the processor 60 based on typical or desired tissue penetration device performance determined through testing at the factory or as programmed in by the operator. The processor 60 may customize by either scaling or modifying the profile based on additional user input information 64. Once the processor has chosen and customized the profile, the processor 60 is ready to modulate the power from the power supply 66 to the penetrating member driver 68 through an amplifier 70. The processor 60 may measure the location of the penetrating member 72 using a position sensing mechanism 74 through an analog to digital converter 76 linear encoder or other such transducer. Examples of position sensing mechanisms have been described in the embodiments above and may be found in the specification for commonly assigned, copending U.S. patent application Ser. No. 10/127,395, (Attorney Docket No. 38187-2551) filed Apr. 19, 2002 and previously incorporated herein. The processor 60 calculates the movement of the penetrating member by comparing the actual profile of the penetrating member to the predetermined profile. The processor 60 modulates the power to the penetrating member driver 68 through a signal generator 78, which may control the amplifier 70 so that the actual velocity profile of the penetrating member does not exceed the predetermined profile by more than a preset error limit. The error limit is the accuracy in the control of the penetrating member.

After the lancing event, the processor 60 can allow the user to rank the results of the lancing event. The processor 60 stores these results and constructs a database 80 for the individual user. Using the database 79, the processor 60 calculates the profile traits such as degree of painlessness, success rate, and blood volume for various profiles 62 depending on user input information 64 to optimize the profile to the individual user for subsequent lancing cycles. These profile traits depend on the characteristic phases of penetrating member advancement and retraction. The processor 60 uses these calculations to optimize profiles 62 for each user. In addition to user input information 64, an internal clock allows storage in the database 79 of information such as the time of day to generate a time stamp for the lancing event and the time between lancing events to anticipate the user's diurnal needs. The database stores information and statistics for each user and each profile that particular user uses.

In addition to varying the profiles, the processor 60 can be used to calculate the appropriate penetrating member diameter and geometry suitable to realize the blood volume required by the user. For example, if the user requires about 1-5 microliter volume of blood, the processor 60 may select a 200 micron diameter penetrating member to achieve these results. For each class of penetrating member, both diameter and penetrating member tip geometry, is stored in the processor 60 to correspond with upper and lower limits of attainable blood volume based on the predetermined displacement and velocity profiles.

The lancing device is capable of prompting the user for information at the beginning and the end of the lancing event to more adequately suit the user. The goal is to either change to a different profile or modify an existing profile. Once the profile is set, the force driving the penetrating member is varied during advancement and retraction to follow the profile. The method of lancing using the lancing device comprises selecting a profile, lancing according to the selected profile, determining lancing profile traits for each characteristic phase of the lancing cycle, and optimizing profile traits for subsequent lancing events.

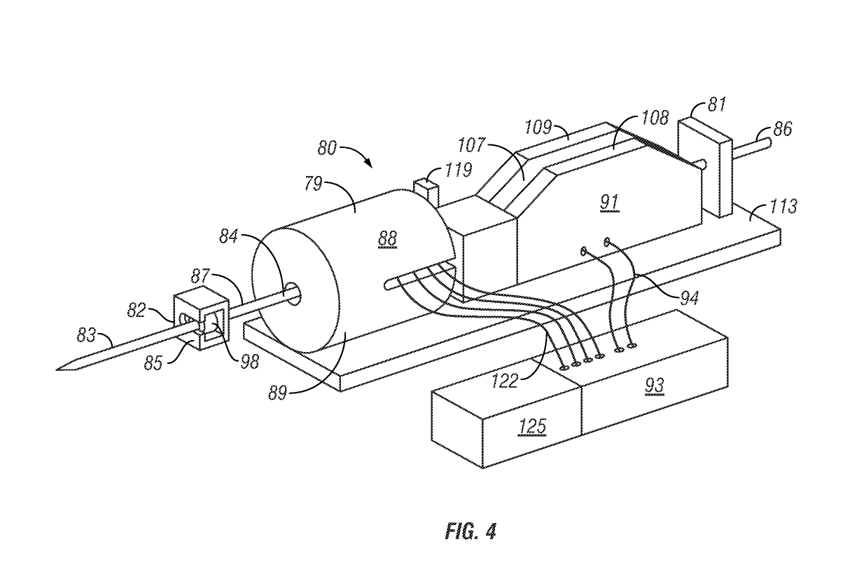

FIG. 4 illustrates an embodiment of a tissue penetration device, more specifically, a lancing device 80 that includes a controllable driver 179 coupled to a tissue penetration element. The lancing device 80 has a proximal end 81 and a distal end 82. At the distal end 82 is the tissue penetration element in the form of a penetrating member 83, which is coupled to an elongate coupler shaft 84 by a drive coupler 85. The elongate coupler shaft 84 has a proximal end 86 and a distal end 87. A driver coil pack 88 is disposed about the elongate coupler shaft 84 proximal of the penetrating member 83. A position sensor 91 is disposed about a proximal portion 92 of the elongate coupler shaft 84 and an electrical conductor 94 electrically couples a processor 93 to the position sensor 91. The elongate coupler shaft 84 driven by the driver coil pack 88 controlled by the position sensor 91 and processor 93 form the controllable driver, specifically, a controllable electromagnetic driver.

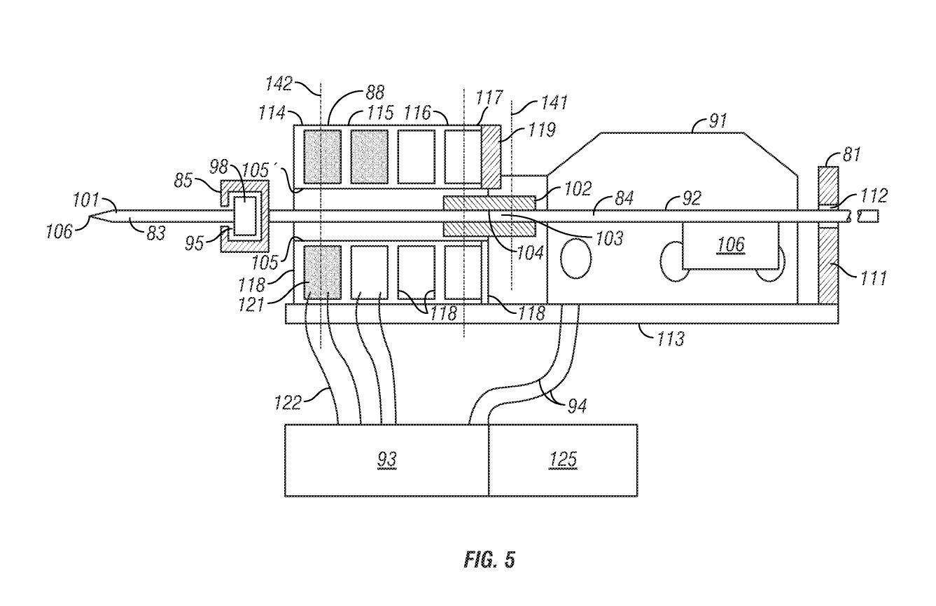

Referring to FIG. 5, the lancing device 80 can be seen in more detail, in partial longitudinal section. The penetrating member 83 has a proximal end 95 and a distal end 96 with a sharpened point at the distal end 96 of the penetrating member 83 and a drive head 98 disposed at the proximal end 95 of the penetrating member 83. A penetrating member shaft 201 is disposed between the drive head 98 and the sharpened point 97. The penetrating member shaft 201 may be comprised of stainless steel, or any other suitable material or alloy and have a transverse dimension of about 0.1 to about 0.4 mm. The penetrating member shaft may have a length of about 3 mm to about 50 mm, specifically, about 15 mm to about 20 mm. The drive head 98 of the penetrating member 83 is an enlarged portion having a transverse dimension greater than a transverse dimension of the penetrating member shaft 201 distal of the drive head 98. This configuration allows the drive head 98 to be mechanically captured by the drive coupler 85. The drive head 98 may have a transverse dimension of about 0.5 to about 2 mm.

A magnetic member 102 is secured to the elongate coupler shaft 84 proximal of the drive coupler 85 on a distal portion 203 of the elongate coupler shaft 84. The magnetic member 102 is a substantially cylindrical piece of magnetic material having an axial lumen 204 extending the length of the magnetic member 102. The magnetic member 102 has an outer transverse dimension that allows the magnetic member 102 to slide easily within an axial lumen 105 of a low friction, possibly lubricious, polymer guide tube 105′ disposed within the driver coil pack 88. The magnetic member 102 may have an outer transverse dimension of about 1.0 to about 5.0 mm, specifically, about 2.3 to about 2.5 mm. The magnetic member 102 may have a length of about 3.0 to about 5.0 mm, specifically, about 4.7 to about 4.9 mm. The magnetic member 102 can be made from a variety of magnetic materials including ferrous metals such as ferrous steel, iron, ferrite, or the like. The magnetic member 102 may be secured to the distal portion 203 of the elongate coupler shaft 84 by a variety of methods including adhesive or epoxy bonding, welding, crimping or any other suitable method.

Proximal of the magnetic member 102, an optical encoder flag 206 is secured to the elongate coupler shaft 84. The optical encoder flag 206 is configured to move within a slot 107 in the position sensor 91. The slot 107 of the position sensor 91 is formed between a first body portion 108 and a second body portion 109 of the position sensor 91.

The slot 107 may have separation width of about 1.5 to about 2.0 mm. The optical encoder flag 206 can have a length of about 14 to about 18 mm, a width of about 3 to about 5 mm and a thickness of about 0.04 to about 0.06 mm.

The optical encoder flag 206 interacts with various optical beams generated by LEDs disposed on or in the position sensor body portions 108 and 109 in a predetermined manner. The interaction of the optical beams generated by the LEDs of the position sensor 91 generates a signal that indicates the longitudinal position of the optical flag 206 relative to the position sensor 91 with a substantially high degree of resolution. The resolution of the position sensor 91 may be about 200 to about 400 cycles per inch, specifically, about 350 to about 370 cycles per inch. The position sensor 91 may have a speed response time (position/time resolution) of 0 to about 120,000 Hz, where one dark and light stripe of the flag constitutes one Hertz, or cycle per second. The position of the optical encoder flag 206 relative to the magnetic member 102, driver coil pack 88 and position sensor 91 is such that the optical encoder 91 can provide precise positional information about the penetrating member 83 over the entire length of the penetrating member's power stroke.

An optical encoder that is suitable for the position sensor 91 is a linear optical incremental encoder, model HEDS 9200, manufactured by Agilent Technologies. The model HEDS 9200 may have a length of about 20 to about 30 mm, a width of about 8 to about 12 mm, and a height of about 9 to about 11 mm. Although the position sensor 91 illustrated is a linear optical incremental encoder, other suitable position sensor embodiments could be used, provided they posses the requisite positional resolution and time response. The HEDS 9200 is a two channel device where the channels are 90 degrees out of phase with each other. This results in a resolution of four times the basic cycle of the flag. These quadrature outputs make it possible for the processor to determine the direction of penetrating member travel. Other suitable position sensors include capacitive encoders, analog reflective sensors, such as the reflective position sensor discussed above, and the like.

A coupler shaft guide 111 is disposed towards the proximal end 81 of the lancing device 80. The guide 111 has a guide lumen 112 disposed in the guide 111 to slidingly accept the proximal portion 92 of the elongate coupler shaft 84. The guide 111 keeps the elongate coupler shaft 84 centered horizontally and vertically in the slot 102 of the optical encoder 91.

The driver coil pack 88, position sensor 91 and coupler shaft guide 111 are all secured to a base 113. The base 113 is longitudinally coextensive with the driver coil pack 88, position sensor 91 and coupler shaft guide 111. The base 113 can take the form of a rectangular piece of metal or polymer, or may be a more elaborate housing with recesses, which are configured to accept the various components of the lancing device 80.

As discussed above, the magnetic member 102 is configured to slide within an axial lumen 105 of the driver coil pack 88. The driver coil pack 88 includes a most distal first coil 114, a second coil 115, which is axially disposed between the first coil 114 and a third coil 116, and a proximal-most fourth coil 117. Each of the first coil 114, second coil 115, third coil 116 and fourth coil 117 has an axial lumen. The axial lumens of the first through fourth coils are configured to be coaxial with the axial lumens of the other coils and together form the axial lumen 105 of the driver coil pack 88 as a whole. Axially adjacent each of the coils 114-117 is a magnetic disc or washer 118 that augments completion of the magnetic circuit of the coils 114-117 during a lancing cycle of the device 80. The magnetic washers 118 of the embodiment of FIG. 5 are made of ferrous steel but could be made of any other suitable magnetic material, such as iron or ferrite.

The outer shell 89 of the driver coil pack 88 is also made of iron or steel to complete the magnetic path around the coils and between the washers 118. The magnetic washers 118 have an outer diameter commensurate with an outer diameter of the driver coil pack 88 of about 4.0 to about 8.0 mm. The magnetic washers 118 have an axial thickness of about 0.05, to about 0.4 mm, specifically, about 0.15 to about 0.25 mm.

Wrapping or winding an elongate electrical conductor 121 about an axial lumen until a sufficient number of windings have been achieved forms the coils 114-117. The elongate electrical conductor 121 is generally an insulated solid copper wire with a small outer transverse dimension of about 0.06 mm to about 0.88 mm, specifically, about 0.3 mm to about 0.5 mm. In one embodiment, 32 gauge copper wire is used for the coils 114-117. The number of windings for each of the coils 114-117 of the driver pack 88 may vary with the size of the coil, but for some embodiments each coil 114-117 may have about 30 to about 80 turns, specifically, about 50 to about 60 turns. Each coil 114-117 can have an axial length of about 1.0 to about 3.0 mm, specifically, about 1.8 to about 2.0 mm. Each coil 114-117 can have an outer transverse dimension or diameter of about 4.0, to about 2.0 mm, specifically, about 9.0 to about 12.0 mm. The axial lumen 105 can have a transverse dimension of about 1.0 to about 3.0 mm.

It may be advantageous in some driver coil 88 embodiments to replace one or more of the coils with permanent magnets, which produce a magnetic field similar to that of the coils when the coils are activated. In particular, it may be desirable in some embodiments to replace the second coil 115, the third coil 116 or both with permanent magnets. In addition, it may be advantageous to position a permanent magnet at or near the proximal end of the coil driver pack in order to provide fixed magnet zeroing function for the magnetic member (Adams magnetic Products 23A0002 flexible magnet material (800) 747-7543).

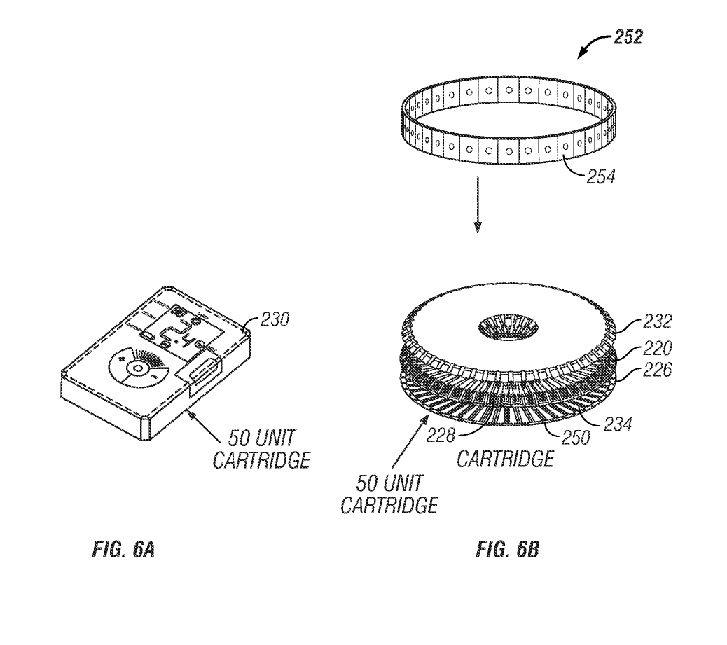

Referring now to FIGS. 6A and 6B, yet another embodiment of the present invention will now be described. It should be understood that this embodiment may be adapted for use with devices described in commonly assigned copending U.S. patent application Ser. No. 10/323,624 (Attorney Docket No. 38187-2608) filed Dec. 18, 2002. FIG. 6A shows a device that may optionally use a cartridge as shown in FIG. 6B. FIG. 6B shows a radial cartridge 220. The cartridge 220 may optionally include a sterility barrier 232 and a substrate 250 having a plurality of analyte detecting members 226. In this embodiment, the cartridge 220 is designed so that blood will enter the fluid chamber 228 and be held there for analysis.

FIG. 6B shows the radial cartridge 220 may optionally be used with a lancing device 230. The radial cartridge 220 may optionally be sealed with a sterility barrier 232 and be coupled to analyte detecting members mounted on a substrate 234. A suitable device is described in commonly assigned, copending U.S. patent application Ser. No. 10/429,196 (Attorney Docket No. 38187-2662) fully incorporated herein by reference for all purposes.

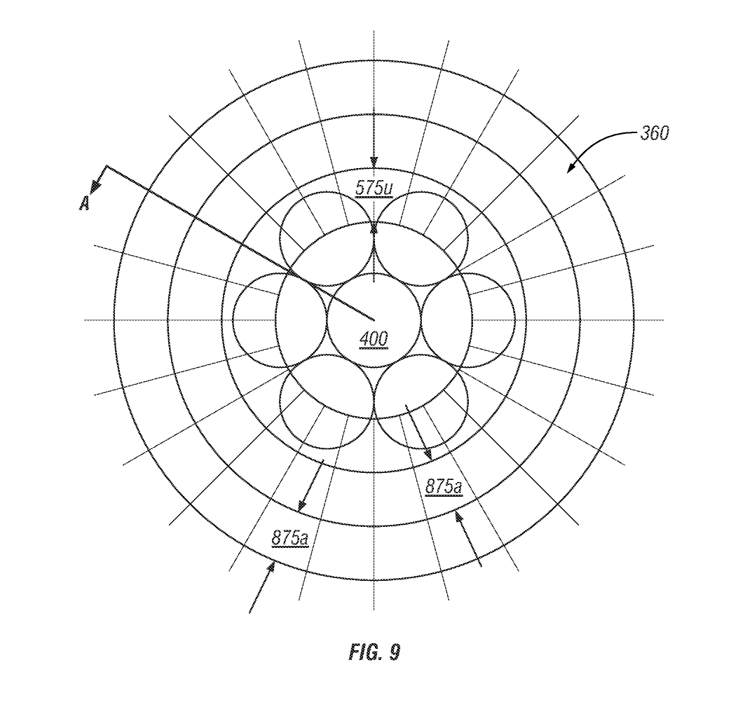

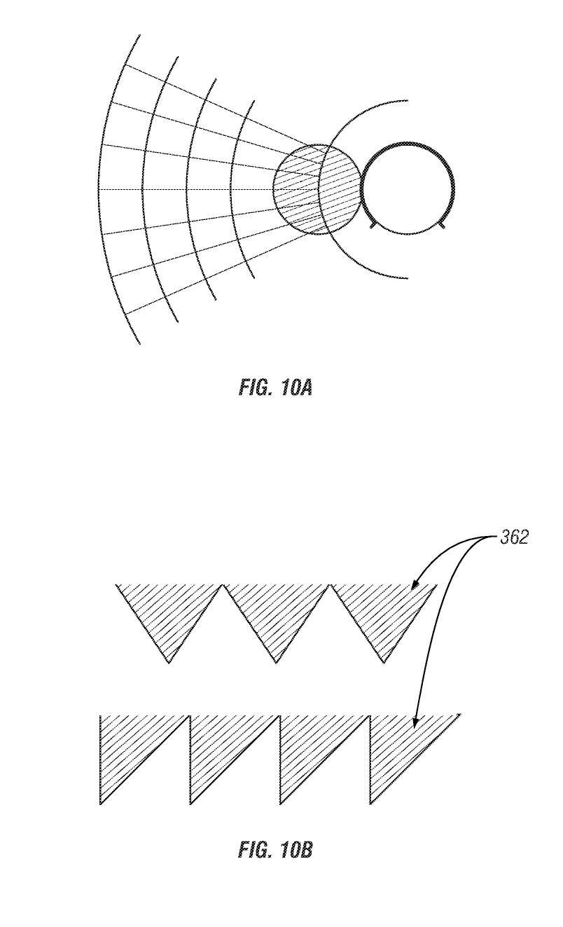

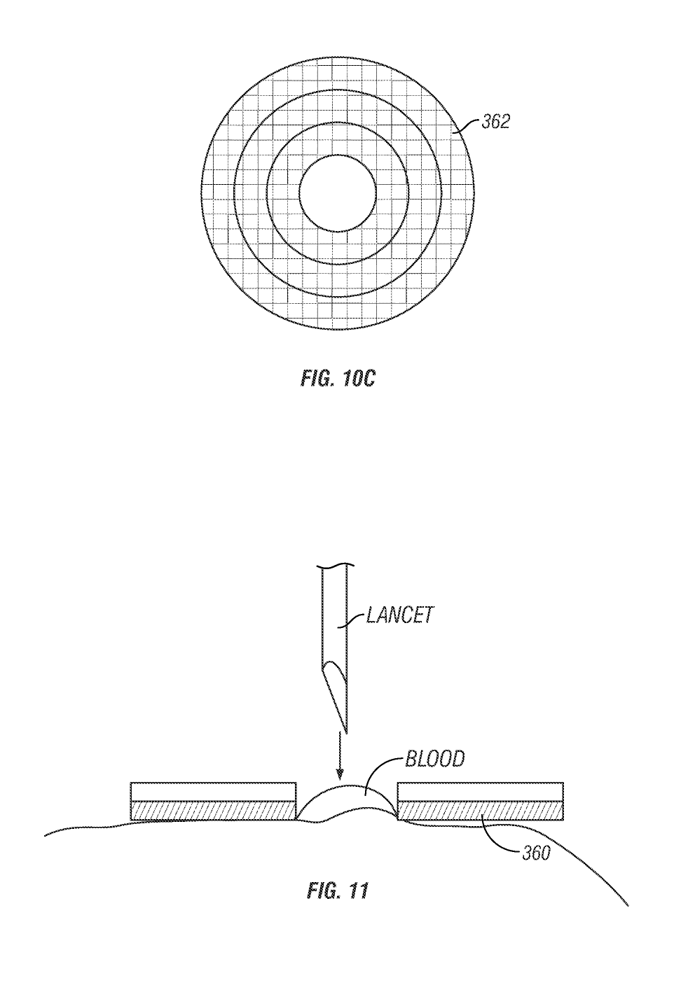

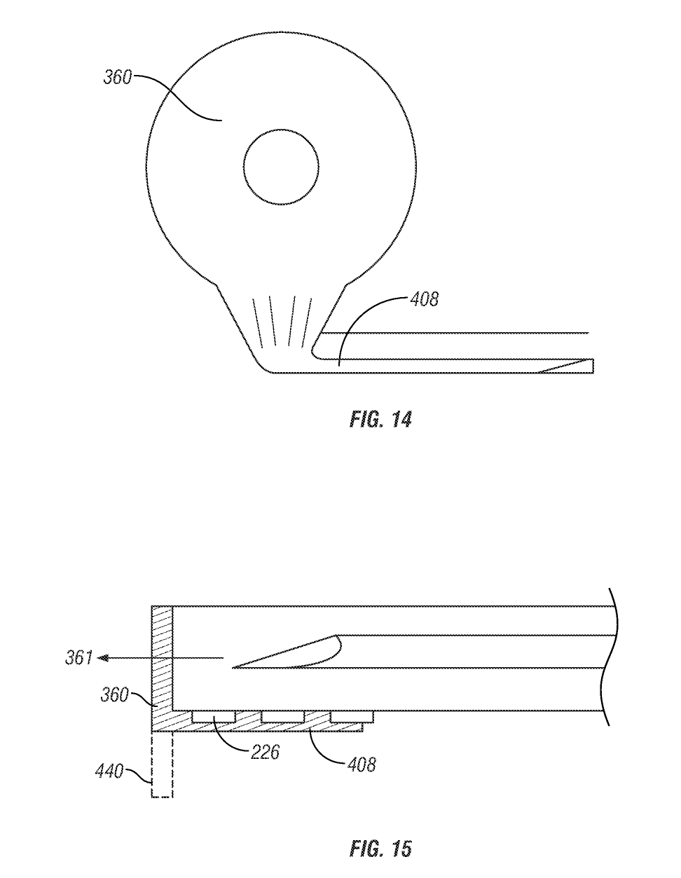

It should be understood that in some embodiments, the layer 234 may be removed and the bottom layer of the cartridge 220 sealed. Instead, a ring 252 with a plurality of analyte detecting members 254 (such as those shown in FIGS. 10A-20) may optionally be in a ring configuration around the penetrating member cartridge 220. This orients one analyte detecting member 254 for each penetrating member in cartridge 220. Some embodiments may optionally have portions of the ring 254 fold underneath the cartridge 220 as shown in FIGS. 14 and 15.

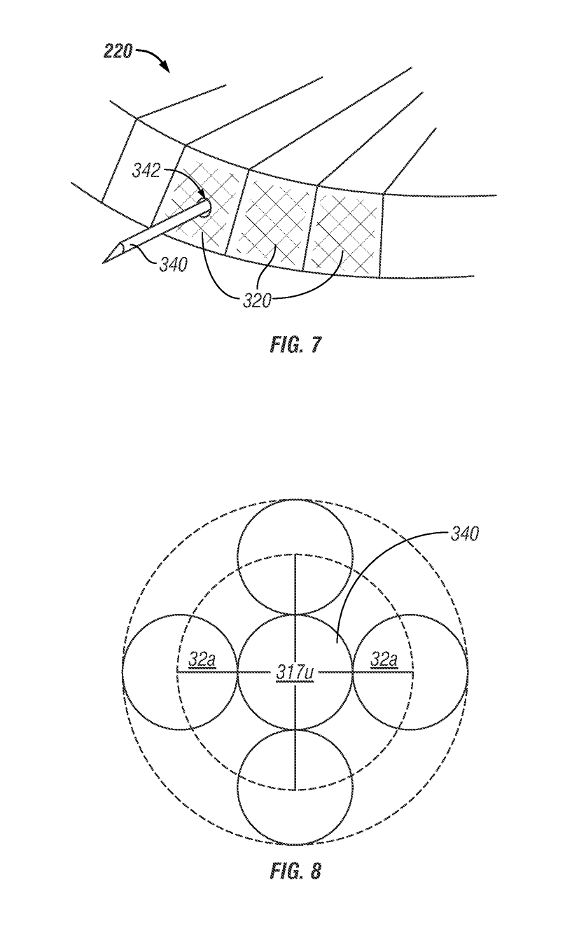

Referring now to FIG. 7, as described above, when a penetrating member 340 is actuated and extends outward from the cartridge 220, the mesh 320 may optionally be pushed aside or pierced by the exiting member 340. The resulting ring of capillary fibers 342 around the wound channel would be available after the penetrating member was retracted to wick the blood sample into the sample channel.

The physical characteristics of the mesh 320 is one aspect for successfully transport of blood to the analyte detecting member 250. In one embodiment, the mesh 320 may be pliable enough the allow relaxation, but maintain contact or near-contact with the skin surface. An active region could be striped on the mesh to allow the blood to only travel in the direction towards the analyte detecting member. A different gauge capillary fiber may optionally be used on the mains versus the cross. In another embodiment, the mains may optionally have a smaller gage and higher pitch to promote vertical movement. As an additional benefit, if the mesh assisted in distributing the force of penetrating member impact with the skin, the cutting efficiency of the penetrating member could be increased.

In another embodiment, the mesh 320 would reduce the amount of micro positioning used to assure that the droplet of body fluid gets to the analyte detecting member. The potential volume required by the analyte detecting member could be reduced by reducing the amount of blood or body fluid that spontaneously rises to the surface of the skin that is either not removed from the skin once the surface tension is released in a traditional, microfluidics methods. Traditional microfluidics could also have a higher volume required to get the blood to the sample chamber.

Referring now to FIG. 8, this embodiment of the present invention pertains to the 100 percent capture of a bodily fluid generated from a wound upon lancing. There are problems when the blood droplet formed immediately after lancing. The droplet can be positioned in any position 360 degrees along the circumference of the lancing location.

Due to the observed low jitter or lateral movement of the penetrating member during the lancing protocol, the fluidic sample capture aperture with mesh will not obstruct the path of the penetrating member. The model of the penetrating member and subsequent droplet formation has provided a geometric dimension that will allow the fluidic sample capture and transport structure to be constructed circumnavigating the entire penetrating member.

This penetrating member circumnavigating sample and capture mesh structure will allow the capture of a produced droplet and transport it directly to the sensor measurement devices.

As seen in FIG. 8, the drawing shows a calculation of the aperture opening based upon the penetrating member 340 diameter and both the observed and specified penetrating member lateral motion resolution. In addition, the aperture ring contains a collection of fluid channels, with respect to this particular disclosure, the mesh is to transport the captured bodily fluid to the measurement sensors which also circumnavigate the aperture opening.

This embodiment of the invention provides a sample, capture, and transport solution to that of an integrated physiological measurement device, which allows the capture of the fluidic sample by mesh immediately upon the penetrating member operation. As seen in FIG. 9, the structure contains an aperture ring structure 360, which surrounds or circumnavigates the penetrating member wound. Upon the release of the bodily fluid from the penetrating member wound, the bodily fluid droplet grows until comes in contact with a portion of the fluid transporting mesh 360. Upon contact with the fluid mesh, the bodily fluid through capillary action is wicked into the capillary mesh and brought forth to the sensors also contained in the aperture ring structure. In one embodiment, the mesh 360 takes the blood and distributes it over a uniform surface.

There is insignificant amount of sucking, pumping, or capillary force. In one embodiment, the mesh 360 spread the blood until the fluid contacts a capillary channel and at that point, the pulling an sucking begins. This is step one spreading. Step two is a partial capillary or some pumping or sucking action (this is the pumping action since there are side walls that are now pulling). Step 3 is taking through a 90 degree bend to bring the fluid to the analyte detecting member.

FIG. 10A shows a close up of a portion of the mesh. FIG. 10B shows that grooves or gratings 362 may also be used to serve the spreading function described. Such grooves may optionally be pressed and create striations on a plastic surface. It is creating a fine textured surface to distribute fluid. FIG. 10C shows the scoring or grooves used to spread the materials.

The mesh 360 or the gratings serves as the initial capture up front, which direct blood to a capillary channel. It is also desirable in some embodiments to transport the blood quickly, hence it is desirable to engage the blood in whatever orientation it may be coming off of the penetrating member. Mesh also displaces volume and thus it will use a lower volume of blood during transport. Single and double meshes can be used. In the present invention, since this is an integrated device, the user is blind as to where the blood droplet is on the penetrating member. It can be in a variety of orientations and the present mesh 360 that surrounds the exit port will capture the blood and lead it to transport.

Regardless of where the blood droplet is, it will be transported. In one embodiment, it takes less than 10 seconds to transport blood to the analyte detecting member. In one embodiment, it takes less than 5 seconds to transport blood to the analyte detecting member.

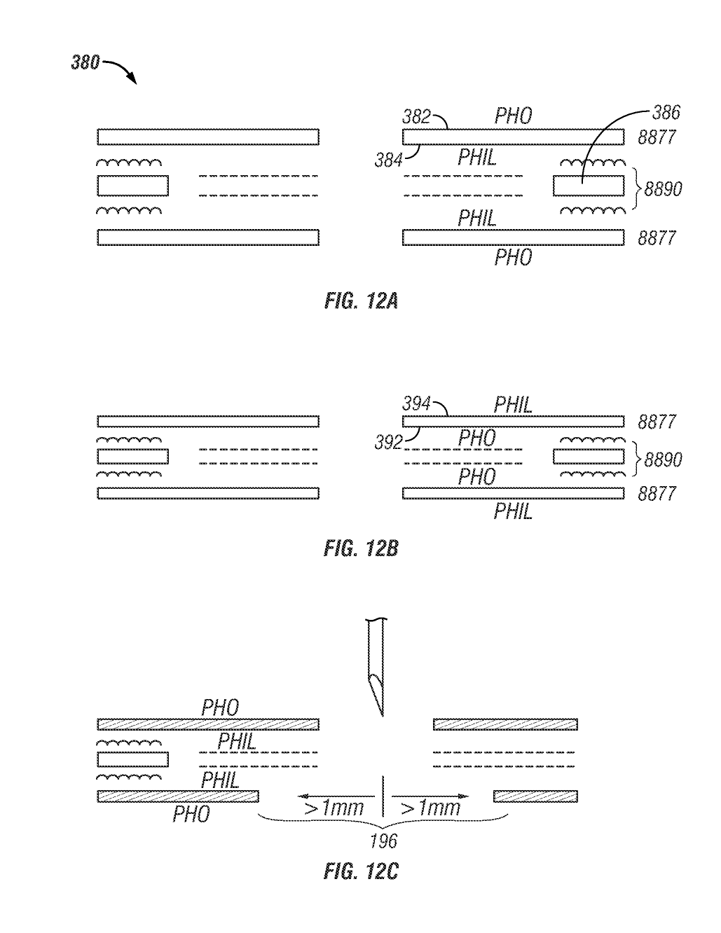

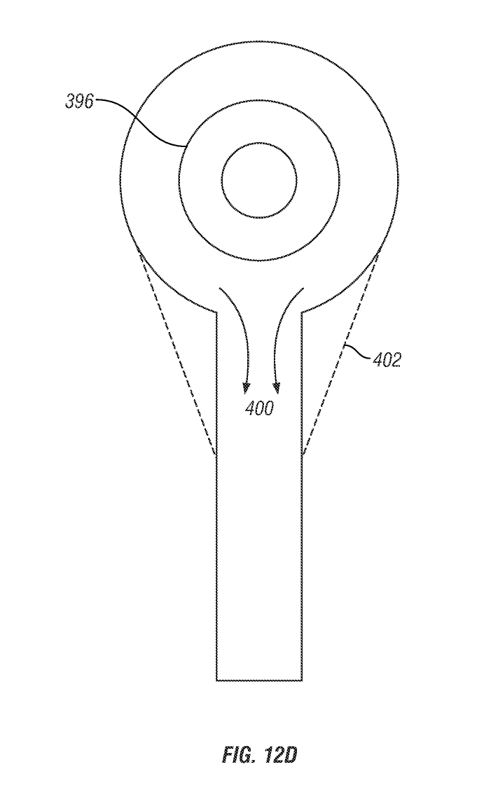

FIG. 11 shows that the blood coming out will contact a mesh 360, regardless of the orientation of the blood on the penetrating member. This surrounding mesh helps to ensure capture. Referring now to FIGS. 12A-12C, the drawings shown describe several configurations, of which there are three, built and tested. The structure in FIG. 12A is one embodiment with a cross section of a fluidic structure 380 with a channel totally free of adhesives. The topside connecting sections comprise of a PET film hydrophobic on the outer most layer 382 and hydrophilic on the inner layer 384 abutting against the hydrophobic double-sided adhesive layer 386. The bottom side would comprise of a PET film hydrophilic on the inner layer abutting against the hydrophobic adhesive and hydrophobic on the outside. The inner fluidic channel region would be a sandwich structure of top PET film/fluidic mesh structures/and bottom PET film. The PET surfaces abutting the mesh structures would be hydrophilic.

The structure in FIG. 12B is a cross section of a fluidic structure with a channel free of adhesives. The structure 390 is very similar to the structure previously described.

However, the difference is in the surface energy of the top and bottom PET films. The hydrophobic surface 392 and hydrophilic surfaces 394 are reversed such that the outer surface is hydrophilic and the inner surface abutting either the adhesive layer or mesh is hydrophobic. The fluidic channel regions remain free of adhesive.

The structure in FIG. 12C is a cross section of a fluidic structure with a channel totally free of adhesives. The structure is very similar to the first structure previously described. However, this structure also incorporates a fluid entry port 396 of which the surface directly facing the droplet of fluid has been slightly oversized in order to expose additional mesh material. There exist a smaller hole on one PET film surface which matches the hole size of the mesh and a larger dissimilar hole on the opposite sandwiching PET film surface.

FIG. 12D shows a front view of the embodiment of FIG. 12C. The blood will be spread and then pulled in the direction indicating by arrows 400. Some embodiments may optionally have a tapered configuration (shown by phantom line 402) and facilitates flow around a 90 degree bend. The taper accounts for bulging or bunch of materials when the neck is bent, which narrows the effective channel available for fluid flow.

These embodiments of this invention entail a method of improving fluidic flow through fluidic mesh transport structures by moderating the selection of hydrophobicity or hydrophilicity through surface energy. This method of moderating or modifying surface energies can be done through a number of different means known to those practicing the arts.

There are a number of options that can be used to treat surfaces to obtain a particular surface preference for degree of hydrophilic or hydrophobic. The concerns relating to the selection of the preferred method of treating a surface depends upon the window of need for this respective treatment. If the window of preference were for a reliable long-term state, then the method may dictate that the bulk properties of the structured material or a physical coating that has good longevity be selected. If the window of preference were to be a short-term state, such as that used in the application of an adhesive, then the method of only treating the surface will be preferred.

The metrology for determining the state of the surface is usually the measurement of the contact angle of a small liquid standard and the material relative to ambient air. The measurement and monitoring of this contact angle and surface energy of time is critical in determining the relative effectiveness of the surface state treatment or bulk fabrication.

The methods of treatment are but are not limited to: a). The fabrication with a natural bulk material used to determine the material's bulk surface properties and the entire process used to fabricate the material. An example of this would be the treatment of PET (Poly (ethylene terephthalate)) or raw polyester. b). The design of the material's surface texture pattern by fabrication processes in conjunction with the material's natural bulk properties. Physical molding or mechanical machining processes may accomplish this. An example of this would be the modification of Young's equation presented later in this discussion. c). The use of high energy sources such plasmas, ion guns, and sputtering techniques to either texture or modify the surface molecular structure. This would include vacuum ion milling, vacuum or argon plasmas, or atmospheric plasmas or corona processes. An example of this would be Argon plasma, Oxygen plasma, ion milling, or Tantec corona treatments. d). The use of wet chemicals to etch and texture the surface molecular structure.

An example of this would be Tetra-Etch. e). The use of thin polymer films deposited by physical vacuum methodologies, spin on coatings, vapor deposited methods, or wet deposited then activated via photonic treatments to actively link molecules of choice for the surface. An example of this would be films by Surmodics. f). The use by design and selection of membrane structures that require the insert or adhesion of films on to surfaces as to create the actual fluid conduction path. An example of this would be membrane films offered by Millipore or paper films offered by Scheicher & Schuell or Sefar America.

A Brief Discussion On Surface Energy of Polymers Wettability and repellency of polymers against water are basic surface properties of the polymers. Hydrophilic and hydrophobic surfaces are results of interactions at an interface between polymer and water layers and closely related to the surface energy of the polymers. Hydrophilic surface means strong interactions with water, and polar groups have to exist at the surface of the polymer. As a result, the contact angle of the polymer against water is small. If the surface energy of the polymer is more than that of water (72.8 mJ/N), the surface of the polymer will contact immediately with water, and the contact angle will be zero. A hydrophobic surface means weak interactions with water at an interface, and the surface consists mainly of non-polar groups. The contact angle of the polymer against water is as large as 90 degrees, in some cases more than 100 degrees.

The surface energy of a material is the excess energy per unit area due to the existence of the free surface. In liquids, the surface energy is conventionally called surface tension. When two different surfaces contact each other and the two surfaces are not mixed, the contact produces an interface and the excess energy is generated at the interface by the formation of the interface. The excess energy per unit area is called interfacial energy or interfacial tension. The contact angle of the polymer against water is a balance among the surface energy of the polymer (Ys) and of water (Yl) and the interfacial energy (Ysl).

The balance of the equation is written Yl COS theta=Ys−Ysl Therefore, the higher the surface energy of the polymer is and the lower the interfacial energy is, the lower the contact angle is. In the extreme case that Ys is equal to Yl and Ysl is zero, the contact angle becomes zero, and complete wetting is accomplished.

The surface energy of the polymer defined by the excess energy per unit area due to the existence of the free surface is closely related to cohesive energy density of the polymer chains. Three methods are proposed for estimation of the surface energy of polymers: 1). The method from the contact angles of polymer against different liquids using Ys=Yl (l+cos theta) ̂2/(4 phî2) phi=(4 (VsVl) ̂ (⅓))/(((Vŝ (⅓))+(Vl̂(l/3))) ̂2 where Vs and Vl are molar volumes of the polymer and the liquid, respectively.

2). The method from the Zisman plat-theoretically, the estimated value is not the real surface energy value 3). The method from the surface tension of melted polymers.

The above discussions provide the basis and foundation of how surface energy on films and meshes can be both moderated and measured. The structures in this invention disclosure concern the creation of circular or rectangular tubular structures and how the fluidic flow might be moderated or enhanced by the use of surfaces modified or moderated by the fore mentioned techniques. The three structures were fabricated and tested. However, the last structure or bottom structure provided the best wicking and attraction of fluid to the structure surface and transport into the fluid channel. The combination of the hydrophilic surfaces abutting the hydrophilic mesh for both sides of the fluidic channel and the dissimilar hole sizes exposing the hydrophilic mesh against a hydrophilic surface demonstrated excellent fluidic action. Wicking action upon the exposed hydrophilic mesh and combined hydrophilic surface and support structure promoted immediate surface action. The combined hydrophilic channel top and bottom walls along with the capillary action of the hydrophilic mesh supported immediate fluid transport from source to destination.

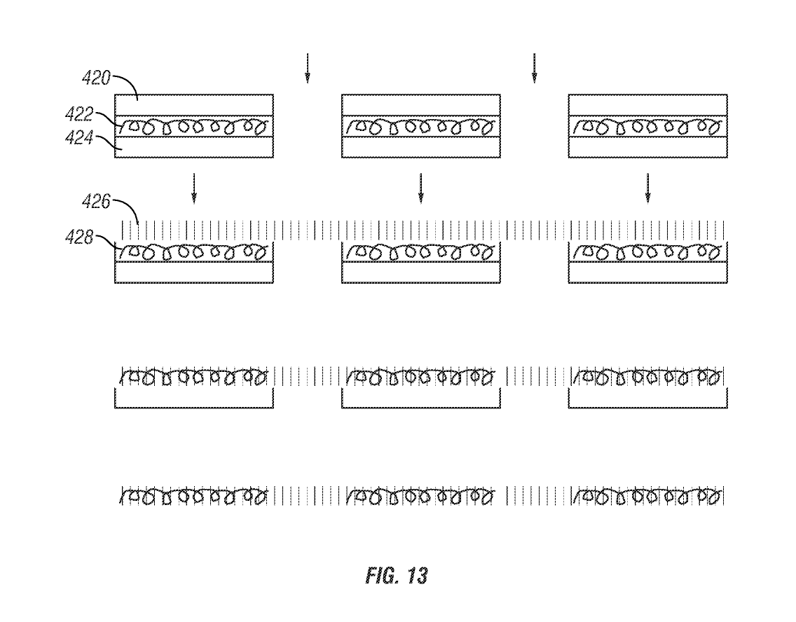

Referring now to FIG. 13, the drawings show a step by step description of the fabrication of one embodiment of an integrated mesh and adhesive structure. The layer by layer assembly is described in the drawings. Another figure at the bottom shows the final assembly of the structure. This invention pertains to the design and fabrication of mesh structures as a method of sample, capture, and transport of bodily fluids. The traditional methods of pattern definition in mesh membrane structures has been to either but and fit the mesh within a predefined physical capillary structure or the impregnating the mesh membrane pores by the process of screen printing.

The process of screen printing involves the use of many different chemicals, light energies, or vapors that might alter the chemistry of the mesh membrane surface chemistry or physics. Thus the use of a prefabricated, preformed, and preprocessed pressure sensitive adhesive to be pressed into the mesh might be the most optimal application for mesh membrane surfaces that are used in medical diagnostics.

FIG. 13 shows one embodiment with a liners 420, an adhesive 422, and another liner 424. Mesh 426 is compressed into adhesive 428. A combination of mesh and adhesive is shown on top of liner. This embodiment of the invention adheres to the principal of using hydrophilic/hydrophobic surface tension. In some embodiment, the adhesives are used to define the channels. Both adhesives are hydrophobic to minimize delamination of the films. The adhesives may optionally be die cut to shape. This facilitates integration of manufacturing. The devices may optionally be hybrid structures using wicking material for capture and then a capillary structure for transport. The mesh leads a little into the capillary and then the fluid just flows. FIG. 14 shows such a mesh 360 leading partially into a capillary structure 408. FIG. 15 shows a side view with the electrodes 226 located over capillary structure 408. This an L-shaped configuration.

Some embodiments may not have a L-bend and may be linear configuration that is vertical as indicated by phantom lines 440. FIG. 15 also shows that the wicking member is oriented to be perpendicular to the path of the penetrating member indicated by arrow 361. The wicking member is oriented to intersect the path of the penetrating member indicated by arrow 361.

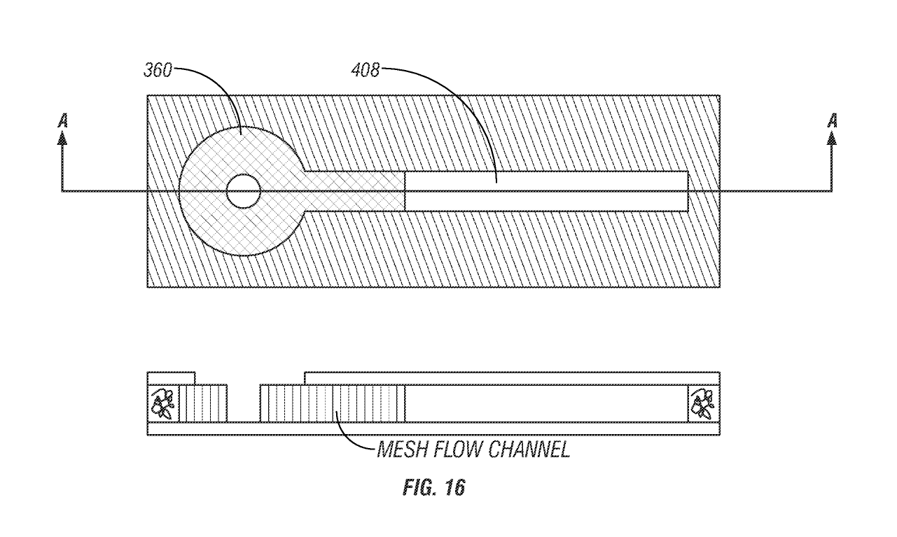

Referring now to FIG. 16, the drawing shows a schematic top and side view depicting the integrated mesh membrane and capillary structure. This embodiment of the invention relates to the integration of a mesh membrane sample and capture structure with a capillary transport to insure stable glucometric measurement. The structure is useful to an integrated sample capture, transport, and measurement device for reliable and accurate performance with very small sample volumes.

This embodiment of the invention pertains to the design and development of a blood droplet sample capture, blood fluid transport, and delivery onto a glucose measurement device. The sample and capture mesh membrane mechanism guarantees consistent capture of a droplet after a penetrating member procedure. The resulting blood droplet from the digit tip is captured by the mesh membrane structure 360 and transported via the mesh membrane mechanism into a small capillary structure 408 consisting of the prior membrane structure less the mesh membrane onto the surface of the glucose measurement device. The height of this cavity for the measurement structure is established by the electrochemistry limitations of the glucose measurement chemistry.

The height specified is known to those practicing the arts. This structure will allow certain sample capture, rapid transport, and reliable measurement. In an electrochemical setup, the electrodes (either a 2 electrode setup or a 3 electrode setup) will be positioned to sample body fluid in the capillary structure area 408.

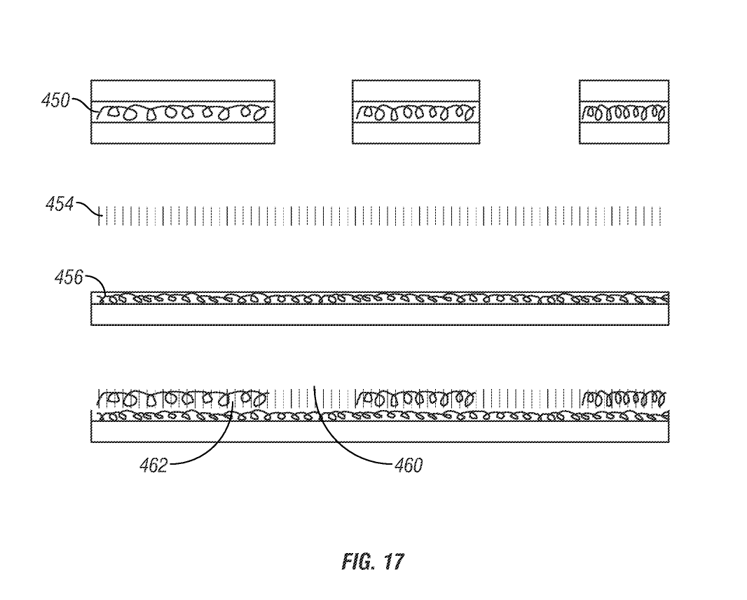

Referring now to FIG. 17, the drawing shows a step by step description of one embodiment for the fabrication of an integrated mesh and adhesive structure. It should be noted that the additional layer of a hydrophilic adhesive layer at the bottom of the mesh membrane provides an excellent sample capture surface within the fluid channel and at the same time augmenting the channel sealing and definition at non fluidic flow regions by design. FIG. 17 shows a hydrophobic adhesive layer 450 between two liners. The device may also have a mesh layer 454. There may optionally be a hydrophilic adhesive layer 456. After assembly, the device will have fluid channels 460 and non-channel regions 462.

This embodiment of the present invention relates to the integration of hydrophobic and hydrophilic adhesives onto and within a mesh membrane for the enhancement of fluidic capture and transport flow. The developed surface energy properties of specific adhesive formulations has allowed the availability of extreme hydrophobic and hydrophilic properties and various viscosities as to promote absorption into the pores of the mesh membranes. Through proper mixing by design, the masking of mesh membranes has been obtainable with pressure sensitive adhesives along with fluid attractive properties to direct optimal fluid capture, transport, and flow.

This embodiment of the present invention may also pertain to the design and fabrication of mesh structures as a method of sample, capture, and transport of bodily fluids. The traditional methods of pattern definition in mesh membrane structures has been to either but and fit the mesh within a predefined physical capillary structure or the impregnating the mesh membrane pores by the process of screen printing.

The process of screen printing involves the use of many different chemicals, light energies, or vapors that might alter the chemistry of the mesh membrane surface chemistry or physics. Thus the use of a prefabricated, preformed, and preprocessed pressure sensitive adhesive to be pressed into the mesh might be the most optimal application for mesh membrane surfaces that are used in medical diagnostics.

The uniqueness of this embodiment of the invention is the further integration of a selective layer of hydrophilic adhesive onto the mesh membrane fluid channel structure to serve a dual purpose of sealing the fluid channel structure from lateral flow leaks and at the same time serve as an enhancement surface for the fluid and transport channel structure.

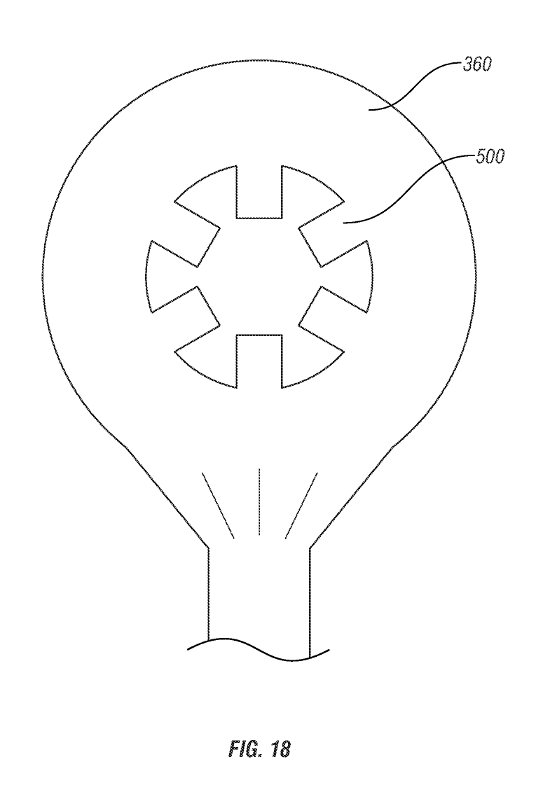

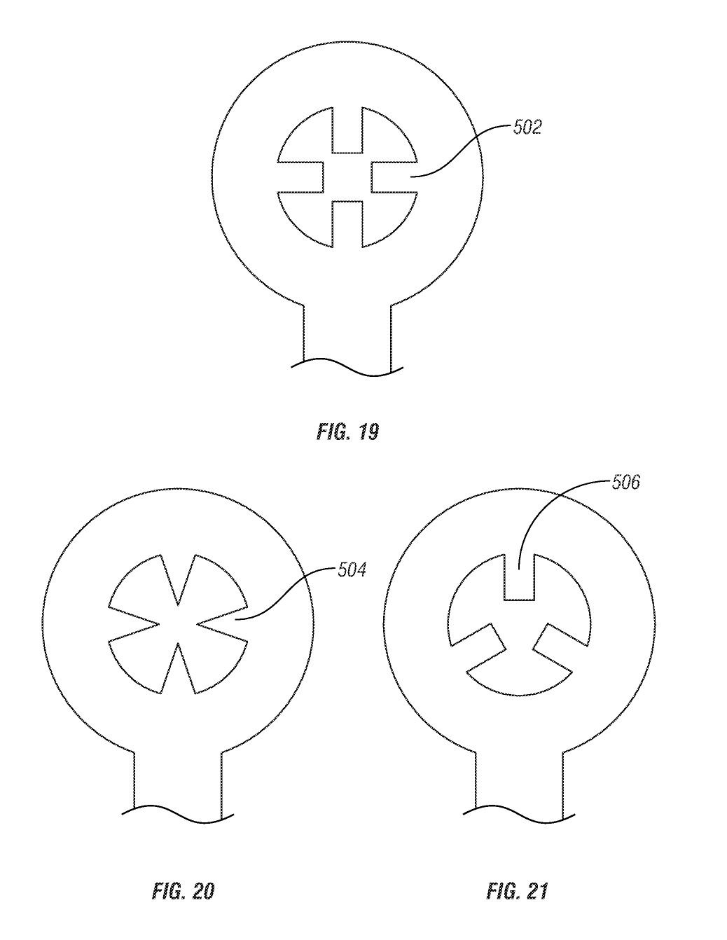

Referring now to FIG. 18 a still further embodiment of the present invention shows that the wicking material may optionally be designed to have flaps which only substantially surround the penetrating member exit but will still engage blood or other body fluid flowing from the wound. Other geometries are shown in FIGS. 19-21.

FIG. 19 shows one embodiment with four rectangular tabs 502. FIG. 20 shows an embodiment with four triangular tabs 504. FIG. 21 shows an embodiment with three rectangular tabs 506. These tabs are positioned to contact body fluid that may be expressed from a wound on the patient. It should be understood that a variety of other shapes, combinations of shapes, combination of shapes described above, and/or other configurations may be used so long as the substantially ensure the blood coming from any orientation from the penetrating member wound will be captured. Some embodiments may simply have a round opening without the tabs. Other shaped openings such as square, rectangular, oval, triangular, octagonal, polygonal, or combinations of any of the above are possible.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

For example, with any of the above embodiments, the location of the penetrating member drive device may be varied, relative to the penetrating members or the cartridge. With any of the above embodiments, the penetrating member tips may be uncovered during actuation (i.e. penetrating members do not pierce the penetrating member enclosure or protective foil during launch). With any of the above embodiments, the penetrating members may be a bare penetrating member during launch. With any of the above embodiments, the penetrating members may be bare penetrating members prior to launch as this may allow for significantly tighter densities of penetrating members. In some embodiments, the penetrating members may be bent, curved, textured, shaped, or otherwise treated at a proximal end or area to facilitate handling by an actuator. The penetrating member may be configured to have a notch or groove to facilitate coupling to a gripper. The notch or groove may be formed along an elongate portion of the penetrating member. With any of the above embodiments, the cavity may be on the bottom or the top of the cartridge, with the gripper on the other side. In some embodiments, analyte detecting members may be printed on the top, bottom, or side of the cavities. The front end of the cartridge maybe in contact with a user during lancing. The same driver may be used for advancing and retraction of the penetrating member.

The penetrating member may have a diameters and length suitable for obtaining the blood volumes described herein. The penetrating member driver may also be in substantially the same plane as the cartridge. In some embodiments, one pin may be configured to contact more than one electrode (such as a U-shaped pin that contacts both the counter and reference electrodes). The driver may use a through hole or other opening to engage a proximal end of a penetrating member to actuate the penetrating member along a path into and out of the tissue. With any of the above embodiments, the strips may have rectangular configurations instead of the lollipop configuration such as that shown in FIG. 12D. It should understood that any of the inventions herein may be used in conjunction or adapted for use with devices disclosed in U.S. Patent Applications Attorney Docket No. 38187-2551, 38187-2608, and 38187-2662. This includes but is not limited to integration of various wicking materials, capillary structures, combinations of the above, or the like with a radial cartridge as described in 38187-2662. The present application is related to U.S. Provisional Application Ser. No. 60/533,981 (Attorney Docket no. 38187-2723).

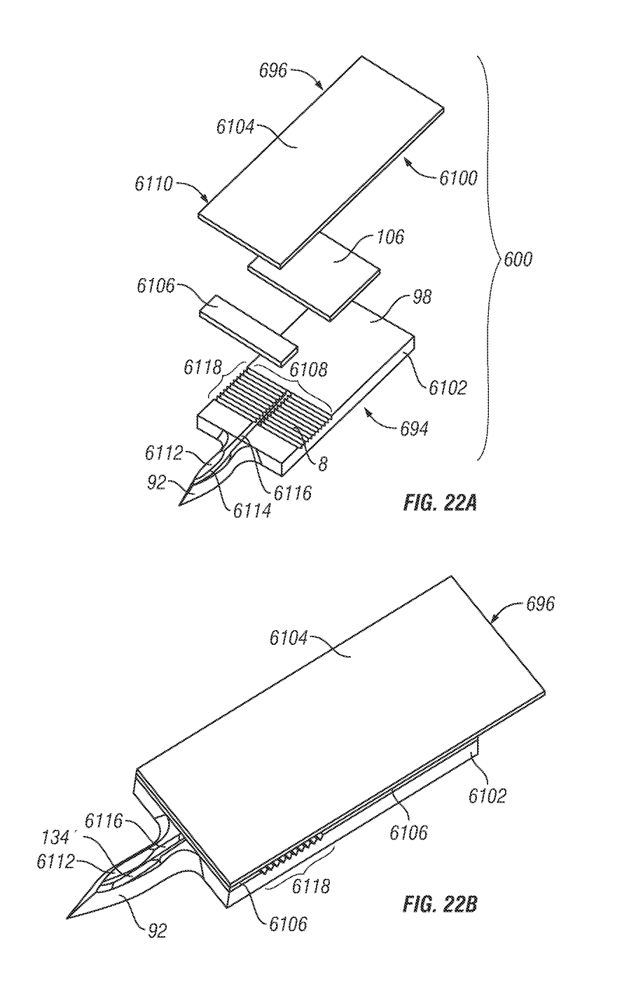

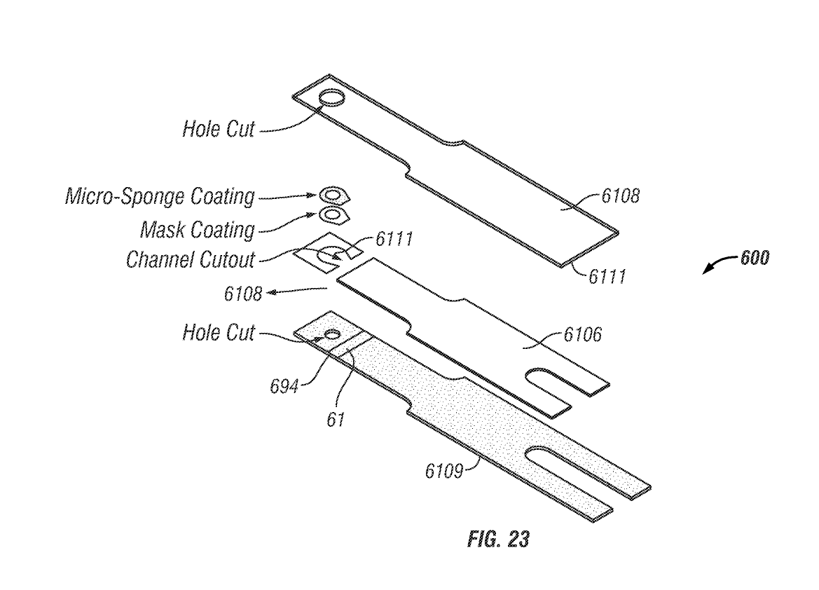

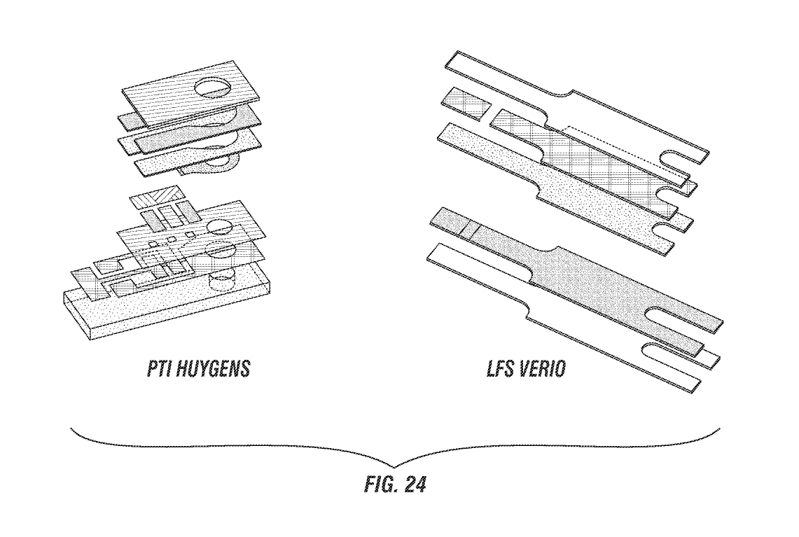

In one embodiment of the present invention, illustrated in FIGS. 22(a) and 22(b), an analyte diagnostic system is provided that uses one or more test strips 600. FIGS. 23 and 24 are exploded views of a test strip 600. The analyte sensor of the test strip may have an electrochemical configuration, or a colorimetric or photometric that is an electrochemical test strip. In any embodiment, the test strip devices and analyte sensors are useful in the determination of a wide variety of different analyte concentrations, where representative analytes include, but are not limited to, glucose, cholesterol, lactate, alcohol, and the like. In many embodiments, the subject test strips are used to determine the glucose concentration in a physiological sample, e.g., interstitial fluid, blood, blood fractions, constituents thereof, and the like.

The test strip 600 can be included in an analyte sensor defined by an electrochemical cell generally having two spaced-apart and opposing electrodes 694 and 696, respectively referred to herein as bottom electrode 694 and top electrode 696, though in use they may oriented in any direction. At least the surfaces of electrodes 694 and 696 facing each other are comprised of a conductive layer 698 and 6100, respectively, such as a metal, deposited on an inert substrate 6102 and 6104, respectively. The spacing between the two electrodes is a result of the presence of a spacer layer 6106 positioned or sandwiched between electrodes 694 and 696. In one embodiment, a micro-sponge coating and a mask coating can be including

In various embodiments, the analyte sensor of the present invention includes a test strip 600 configured to provide, (i) the user with an ability to place its can place its finger on a housing that houses at least a portion of the test strip 600, press a button and obtain an accurate glucose reading; (ii) a one step glucose diagnostic system is provided that has a seamless, automatic series of steps to lance the user's finger, draw blood, capture and transport the blood to a sensor of the test strip 600 and report a result, (iii) one step glucose measurement using sample capture, sample transport and measurement with an electrochemical sensor; (iv) one step glucose measurement that has structures for allowing a lancing event to be conducted, collecting a sample, transporting a sample and measuring the sample; (v) a step glucose measurement with structures for allowing a lancing event to be conducted, collecting a sample, transporting a sample and measuring the sample, where the structures are closely fluidicly coupled, such that a sample, expressed from a lancing event, presents itself at a prescribed location, and the structures enable the collection of this sample and it is subsequently transported to the measurement cell; (vi) a glucose sensor with structures that enables a lancing event, accomplish the sample capture and sample transport functions in a sensor design in one step testing.

In one embodiment of the present invention, a one step analyte diagnostic system is provide that allows a user to place its finger on a housing of the analyte diagnostic system, activate such as by pressing a button and obtain an accurate glucose reading in one single action. This is called bleed to read without additional actions. A seamless, automatic series of steps is used to lance the finger, draw blood, capture and transport the blood to the glucose sensor and then report a result. In one embodiment, sample capture, transport and measurement is done with an electrochemical sensor forming a portion of a reaction zone 6108 of the test strip 600.

In various embodiments, sample capture structures are provided that allow the lancing event to be conducted, along with collecting, transport and measuring an analyte sample in one step. These sample capture structures provide for close fluid coupling in order that an analyte sample obtained following a tissue penetration by a penetrating member through skin, expressed from a lancing event, presents itself at a prescribed location. These sample capture structures enable the collection of the analyte sample and its subsequent transport to the reaction zone 6108 where the analyte sensor resides.

With the present invention, structures and methods are provided that enable a lancing event, accomplish sample capture and sample transport in a sensor design that supports one step testing. In various embodiments, the present invention provides for one step testing by, (i) analyte sample capture layout; (ii) analyte sample capture and transport configurations; (iii) structures of sample capture; (iv) processes for forming sample transport, and the like.

In certain embodiments, the electrodes 694 and 696 are generally configured in the form of elongated rectangular strips but may be of any appropriate shape or configuration. Typically, the length of the electrodes ranges from about 0.5 to 4.5 cm and usually from about 1.0 to 2.8 cm. The width of the electrodes ranges from about 0.07 to 0.8 cm, usually from about 0.20 to 0.60 cm, and more usually from about 0.1 to 0.3 cm. The conductive layers and their associated substrate typically have a combined thickness ranging from about 100 to 500 micrometer and usually from about 125 to 250 micrometer.

Spacer layer 6106 can have a double-sided adhesive to hold the electrodes. The spacer layer is can be cut to provide a reaction zone or area 6108, creating a channel cutout 6111. A redox reagent system or composition can be on the bottom electrode 696 to form an end of a reaction zone 6108, where the reagent system is selected to interact with targeted components in the fluid sample, typically whole blood, during an assay of the sample. Redox reagent system 6110 can be deposited on the conductive layer 6100 of top electrode 696 wherein, when in a completely assembled form, redox reagent system 6110 resides within reaction zone 6108. With such a configuration, bottom electrode 694 serves as a counter/reference electrode and top electrode 696 serves as the working electrode of the electrochemical cell. However, in other embodiments, depending on the voltage sequence applied to the cell, the role of the electrodes can be reversed such that the bottom electrode serves as a working electrode and top electrode serves as a counter/reference electrode.

As mentioned above, electrodes 694 and 696 generally face each other and are separated by only a short distance, such that the spacing between the electrodes is extremely narrow. This minimal spacing is a result of the presence of a spacer layer 6106 positioned or sandwiched between electrodes 694 and 696. The thickness of spacer layer 6106 may range from 10 to 750 micrometer and is often less than or equal to 500 micrometer and usually ranges from about 25 to 175 micrometer. Spacer layer 6106 can have double-sided adhesive to hold electrodes 694 and 696 together. A top substrate 6108 sandwiches in the spacer layer 6106, as more fully described hereafter.

The spacer layer 6106, substrates 6104 and 6109 may be made of a Mylar plastic film. The thickness of an inert backing material can be about 25 to 500 micrometers and usually from about 50 to 400 micrometer. The thickness of the metal layer can be about 10 to 100 nanometer and more particularly from about 10 to 50 nanometer.

In certain embodiments, spacer layer 6106 is configured or cut so as to provide a reaction zone or area 6108, where in many embodiments the volume of the reaction area or zone 6108 can have a volume in the range from about 0.01 to 10 microliters, usually from about 0.1 to 1.0 microliters and more usually from about 0.05 to 1.0 microliters. However, the reaction area may include other areas of the test strip or be elsewhere all together, such as in a fluid pathway, described below in more detail, or the like. Spacer layer 6106 may define any appropriately shaped reaction area, e.g., circular, square, triangular, rectangular or irregular shaped reaction areas, and may further include side inlet and outlet vents or ports.

The present invention provides for body fluid sample capture elements and designs to be included with test strip 600. In certain embodiment, sample capture provides a path that allows that a penetrating member to be intimately in with sample capture fluidics.

The following definitions are used with sample capture of the present invention:

Sample capture layout: The physical layout of the sample capture feature(s), interconnecting transport feature(s) and sensor/reaction zone 6108.

Lancing aperture: The presence of an aperture for a penetrating member to breach for the purposes of enabling a lancing event.

Sample capture aperture: An aperture for the collection of a blood sample expressed from the lancing wound.

Sample transport structure: A structure for the transport of a sample (blood) from the sample capture feature to the sensor/reaction zone 6108, that is the glucose measurement cell.

The sample capture can be a structure that creates a body fluid flow in a surrounding relationship to the penetrating member. In this regard, the sample capture element can be an aperture that provides for body fluid flow around the penetrating member, e.g., a penetrating member aperture. The sample capture mechanism provides for surround a penetrating member lancing wound. In various embodiments, sample capture can be an aperture, include a micro sponge, a hydrophilic coating, a continuous coating, a capillary opening that is located in a way that it meets the requirement, an annular capillary, and the like. For those surfaces where it is desired to not have want, those surfaces can be hydrophobic, or coated with a hydrophobic coating. As a non-limiting example, a top cover can be hydrophobic. Optionally a sample capture can include a transport structure to provide that the blood moves from the sample capture to the within reaction zone 6108/sensor. The sensor is the active electrochemical region, between electrodes 694 and 696. As a non-limiting example, sample capture is in close proximity to the skin. In one specific embodiment, it is about 300 microns.

In one embodiment, sample capture has a horizontal topology. The surface or other topology serves to collect blood from a wound in the body such as the finger. The horizontal structure is typically a planar structure. Because a lancing event creates uncontrolled spontaneity of blood, it is important to have a sample capture geometry/structure that can collect blood, independent of the uncontrolled characteristics of expression. With the present invention, sample capture can be a structure surrounding the lancing wound and in practice, surrounding the penetrating member path. In this embodiment, the characteristics of the sample capture include but are not limited to, to preserve a 360 degree surround of the penetrating member point; other shapes of sample capture structure such as oblong, start, slot and the like; and lancing and blood collection apertures can be made larger by varying structure, potentially easing alignment requirements in manufacturing, and use.

In another embodiment of the present invention, sample capture has a vertical topology with layers, laminations, channel heights and the like. A vertical stack up, or other structure, serves to build the manufactured structure of the sample capture. The vertical structure can be in the form of one or more, channels, layers, laminations, printed structures and the like. The characteristics of the vertical topology are: the sample transport channel can be ‘taller’ than the sensor to have it have relatively less capillary action than the sensor/reaction zone 6108, a barrier layer can be used to prevent blood from reaching, and reacting, with reagent, and is useful in defining the sensor/reaction zone 6108.

In one embodiment of a method for forming sampling transport a vertical stack up or other structure serves to build the manufactured structure of the sample capture. As previously mentioned, the vertical structure can be, the channel, layers, laminations, printed structures and the like. In one embodiment, the process for building a sensor/reaction zone 6108 is as closely tied to the design of the sensor/reaction zone 6108 as are the topologies. Process methods represent the manufacturing process, the interactions of layers or topologies with each other and directly affect all aspects of sensor/reaction zone 6108 performance.

Some of the characteristics of the process include but are not limited to, printing processes such as screen printing, roller printing, pad printing, ink jet (sprayed) printing, and the like; lamination which can be conversion or non-conversion processes, spacer layers, adhesives, cover layers, and the like; different printing processes such as ink jet, roller, slot, mask, needle and the like; kiss cut processes with linear or patterned cuts, differential removal of cut areas to serve as masking for other processes and the like.

A variety of different sample capture materials can be utilized. In one embodiment, a material or surface is provided for collecting expressed blood from a lancing event. In some embodiments a material is used, such as a hydrophilic material with very high capillary action, to facilitate the collection of sample and to make this sample available for transport to the sensor/reaction zone 6108. Some of the characteristics of the sample capture materials include but are not limited to, micro-sponge materials, a hydrophilic layer with a micro structure of small features providing very high capillary action for collecting blood and the like.

A microneedle can be coupled or integrated with the strip 600. As a non-limiting example, a microneedle 692 can be integrally formed with and extend from bottom electrode 694. The microneedle is shown with a space-defining configuration in the form of a concave recess 6112 within its top surface. The recess creates a corresponding space within skin tissue upon penetration of microneedle 692 into the skin. This space acts as a sample fluid collection reservoir wherein fluid released upon penetration is pooled within the space prior to transfer into the electrochemical cell. An opening 6114 to further expose the pooling area defined by recess 6112 to the outside environment may also be included, thereby increasing the volume and flow rate of body fluid into the pooling area.

The analyte sensor device 690 can include a sample fluid transfer or extraction pathway or channel 6116 which extends from recess 6112 to within the sensor/reaction zone 6108. At least a portion of a proximal end of the pathway resides within the sensor/reaction zone 6108 portion of device 690, specifically within reaction zone 6108, also known as the analyte sensor, and a portion of a distal end of pathway 114 resides within microneedle 692. The electrodes 694 and 695, their associated chemistries in reaction zone 6108 are known as the analyte sensor. Pathway 6116 is dimensioned so as to exert a capillary force on fluid within the pooling area defined by recess 6112, and draws or wicks physiological sample to within the reaction zone. Extending laterally from proximal portion 6114 of the pathway to within a portion or the entirety of the reaction zone are sub-channels 6118. The sub-channels facilitate the filling of reaction zone 6108 with the sampled fluid.

A redox reagent system or composition is present at electrode 694 or 696 to form a portion of reaction zone 6108. The reagent system is selected to interact with targeted components in the fluid sample during an assay of the sample. The redox reagent is the chemistry of the sensor/reaction zone 6108. Redox reagent system can be deposited on the conductive layer 6100 of top electrode 696 wherein, when in a completely assembled form, the redox reagent system 14 resides within reaction zone 6108. With such a configuration, bottom electrode 694 serves as a counter/reference electrode and top electrode 696 serves as the working electrode of the electrochemical cell. However, in other embodiments, depending on the voltage sequence applied to the cell, the role of the electrodes can be reversed such that bottom electrode 694 serves as a working electrode and top electrode 696 serves as a counter/reference electrode. In case of a double pulse voltage waveform, each electrode acts as a counter/reference and working electrode once during the analyte concentration measurement.

As non-limiting examples, reagent systems of interest typically include an enzyme and a redox active component (mediator). The redox component of the reagent composition, when present, is made up of one or more redox agents. A variety of different redox agents, i.e., mediators, is known in the art and includes: ferricyanide, phenazine ethosulphate, phenazine methosulfate, pheylenediamine, 1-methoxy-phenazine methosulfate, 2,6-dimethyl-1,4-benzoquinone, 2,5-dichloro-1,4-benzoquinone, ferrocene derivatives, osmium bipyridyl complexes, ruthenium complexes, and the like. In many embodiments, the redox active component of particular interest is ferricyanide, and the like. The enzyme of choice may vary depending on the analyte concentration which is to be measured. For example, suitable enzymes for the assay of glucose in whole blood include glucose oxidase or dehydrogenase (NAD or PQQ based). Suitable enzymes for the assay of cholesterol in whole blood include cholesterol oxidase and esterase.

Other reagents that may be present in the reaction area include buffering agents (e.g., citraconate, citrate, malic, maleic, phosphate, “Good” buffers and the like); divalent cations (e.g., calcium chloride, and magnesium chloride); surfactants (e.g., Triton, Macol, Tetronic, Silwet, Zonyl, and Pluronic); and stabilizing agents (e.g., albumin, sucrose, trehalose, mannitol and lactose).

Referring more specifically to FIGS. 23 and 24, three layers of plastic, including but not limited to Mylar, can be used for strip 600. The bottom layer is substrate 6104 with a covering. In one embodiment, a palladium covering is sputtered on the substrate 6104. Also included are detergents, wetting agents, non-foaming agents and the like, as recited above. The spacer layer 6106 has a slot 6111 in it, which creates capillary flow, and can have pressure sensitive adhesive on both sides. The top substrate 6108 can be made of a plastic and include a conductive material, including but not limited to a gold coating. In one embodiment of the present invention, sample capture structures are positioned in proximity to a flow channel or aperture where an analyte sample travels from a wound created by a penetrating member, to the analyte or reaction zone 6108 of the strip 600. Substrate 6104 includes a conductor, including but not limited to palladium, followed by in a traverse direction electrode 694. Spacer layer 6106 exposes the chemistry 6111, including electrode 694 to an analyte sample.

The top substrate 6108 can include a conductor, including but not limited to a gold plating, which serves as electrode 696. The conductor or gold 6111 coupled to substrate 6109 creates a cavity over the chemistry in the bottom substrate and the reaction zone. It is at this cavity where the analyte fluid is dosed, and it is here where sample capture structures can be coupled.

Referring to FIG. 25, one embodiment of strip 600 has the sample capture positioned adjacent to the sensor/reaction zone 6108, but does not impinge on the sensor/reaction zone 6108. The sample capture has a close fluidic coupling. This embodiment is flexible, is suitable for process constraints for the manufacturing of strip 600 and maintains separation of function for sample capture versus measurement.

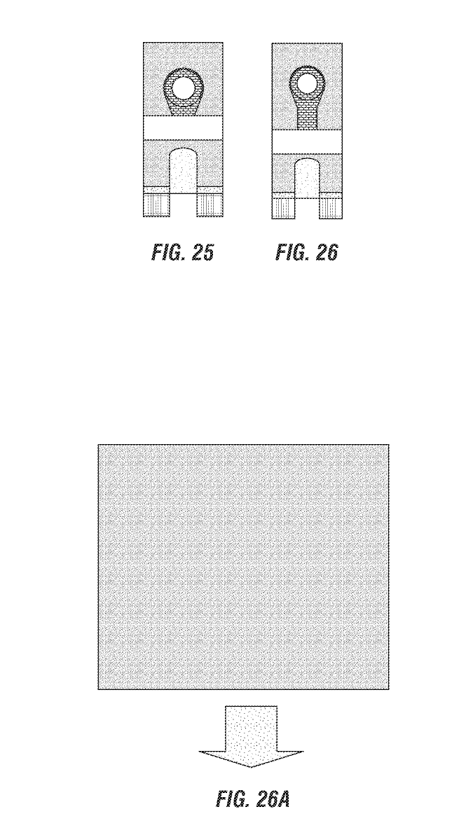

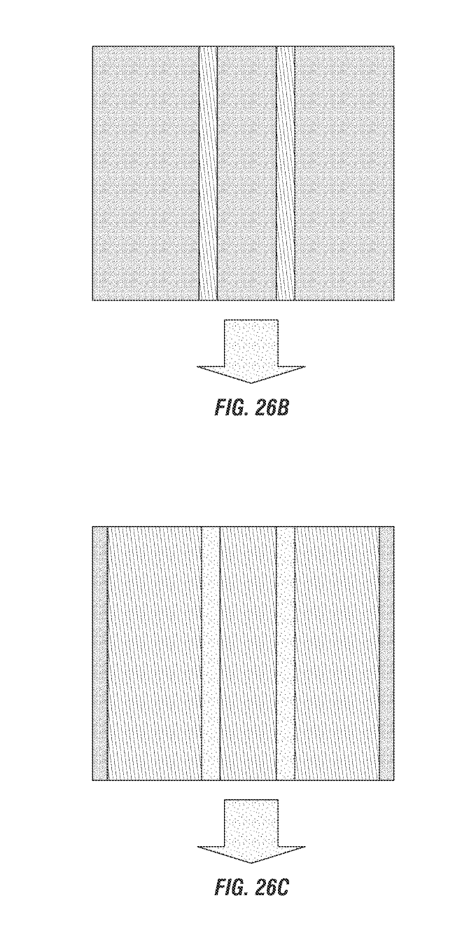

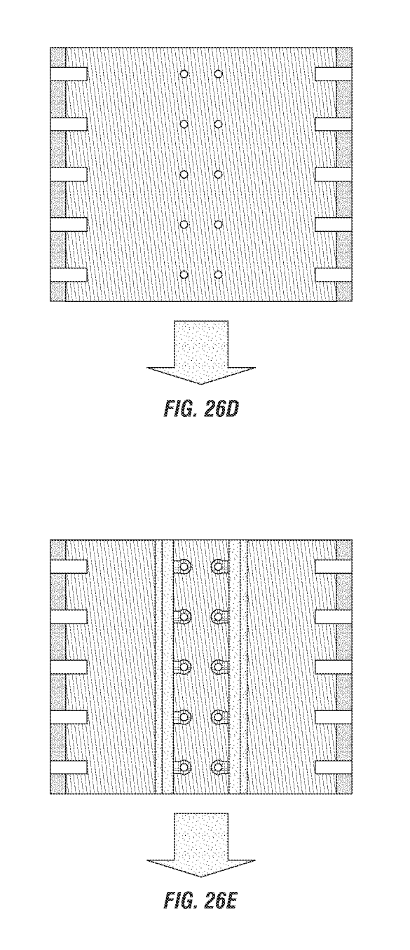

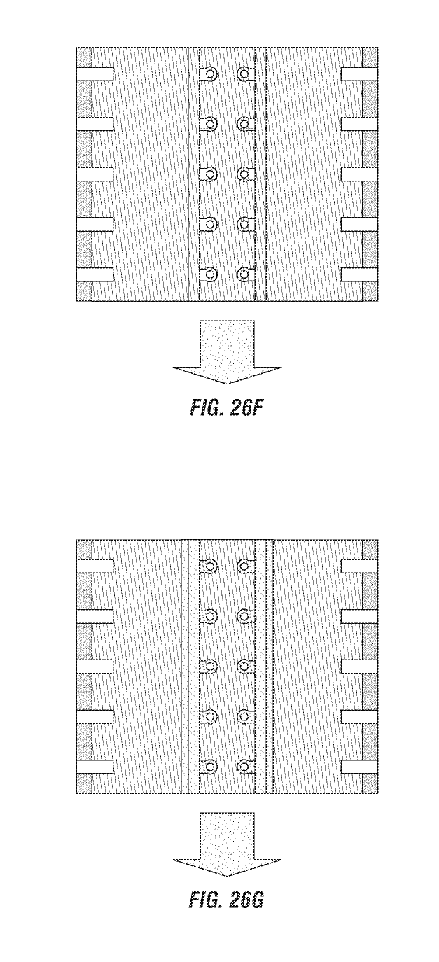

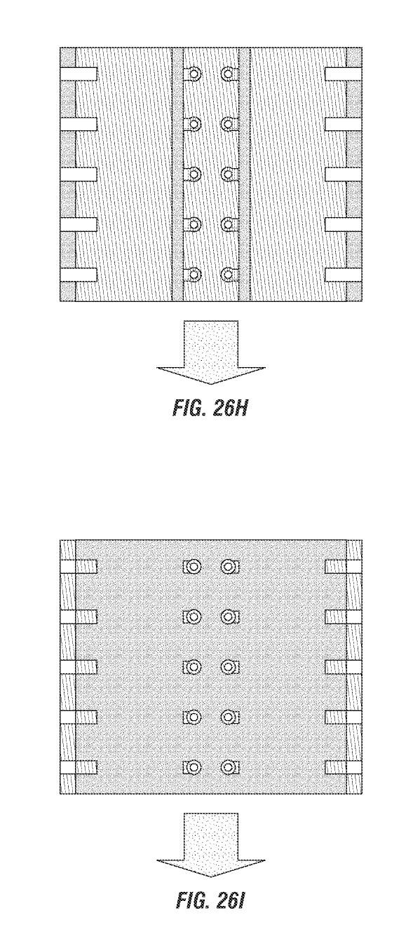

FIG. 26 illustrates an embodiment of a strip 600 with a penetrating member axis that is perpendicular to a plane of the test strip. The FIGS. 25 and 26 embodiments can be made with the process steps illustrated in FIGS. 26(a) through 26(j) with a palladium coated substrate 6104 with surface treatment on a roll.

A slot, or other method, is coated to add reagent chemistry, including but not limited to GDH-FAD w/ mediator. A spacer layer 6106, roll based, is laminated. Substrate and adhesive spacer is punched to feature contact legs and penetrating member aperture. Optionally, a feature for registration of subsequent steps can be added. The spacer layer 6106 is kiss cut, both for sample capture and sensor/reaction zone 6108 area. The spacer area defines a sample capture structure removed and registration to a penetrating member aperture is required. The reagent for the sensor/reaction zone 6108 is still covered by the spacer layer 6106. The objective is to define the sample capture features and provide for the isolation of this feature from the sensor/reaction zone 6108 relative to glucose measurement, as well as to provide the intimate fluidic coupling. The sample capture structure is treated with a blocking layer to eliminate the sample capture structure from being part of active sensor/reaction zone 6108. The blocking layer is in place to ensure that the sample capture and transport features are not part of the sensor/reaction zone 6108 volume or active area.

The sample capture structure is treated with a micro-sponge layer. The sensor/reaction zone 6108 is defined with the kiss cut spacer layer 6106 removed. This exposes the reagent. A gold cover layer is applied which may require registration. The roll is cut to singulate the single strips 600 as a single, ribbon, block and the like.

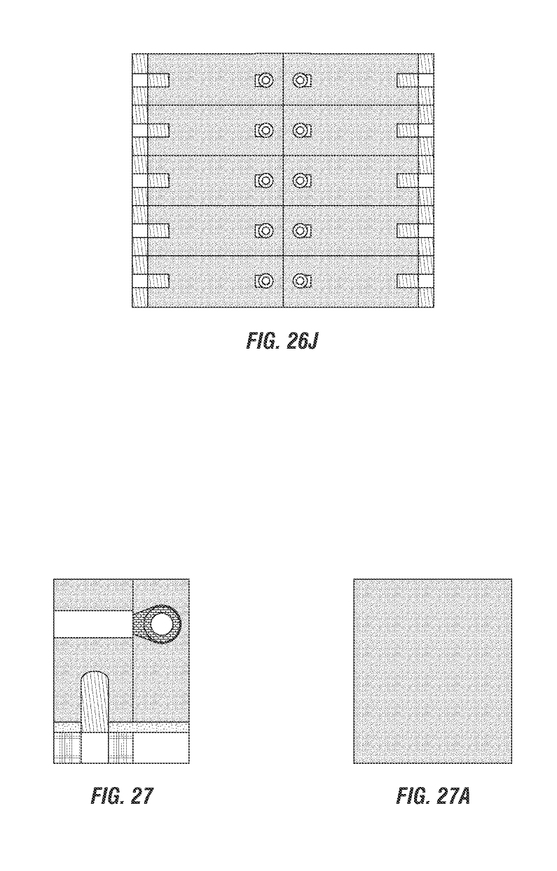

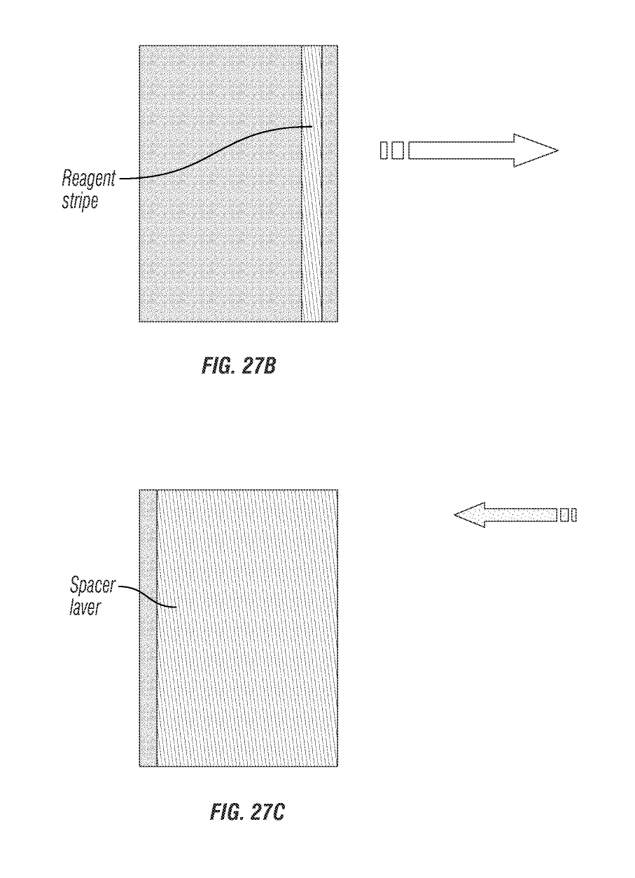

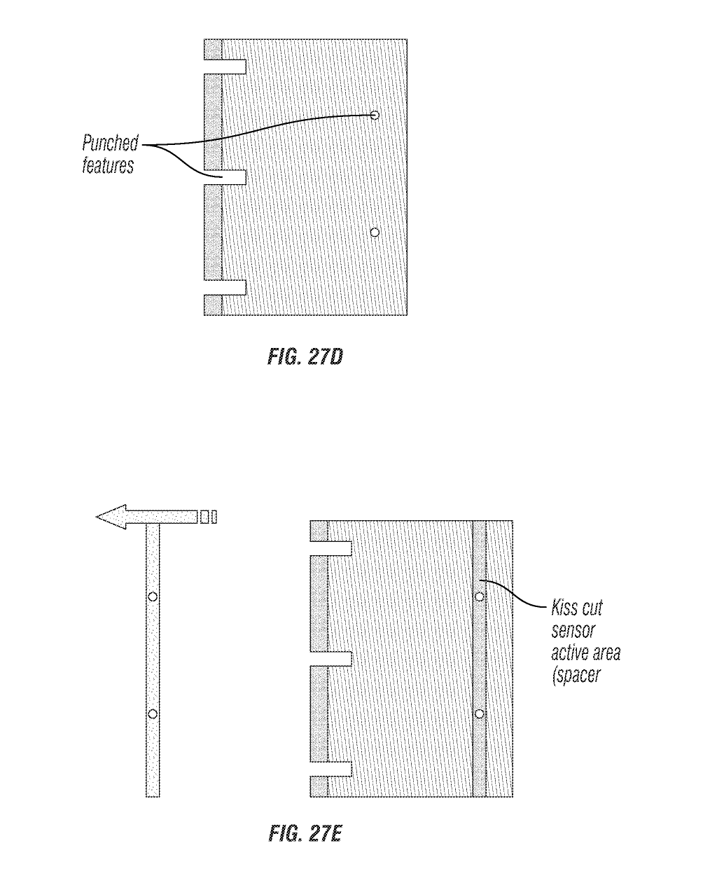

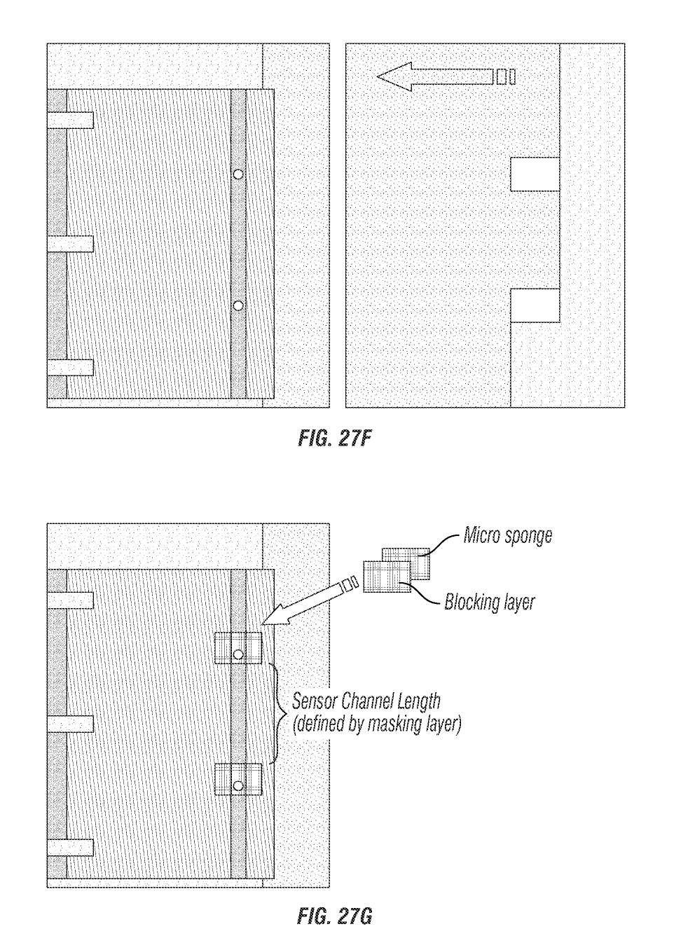

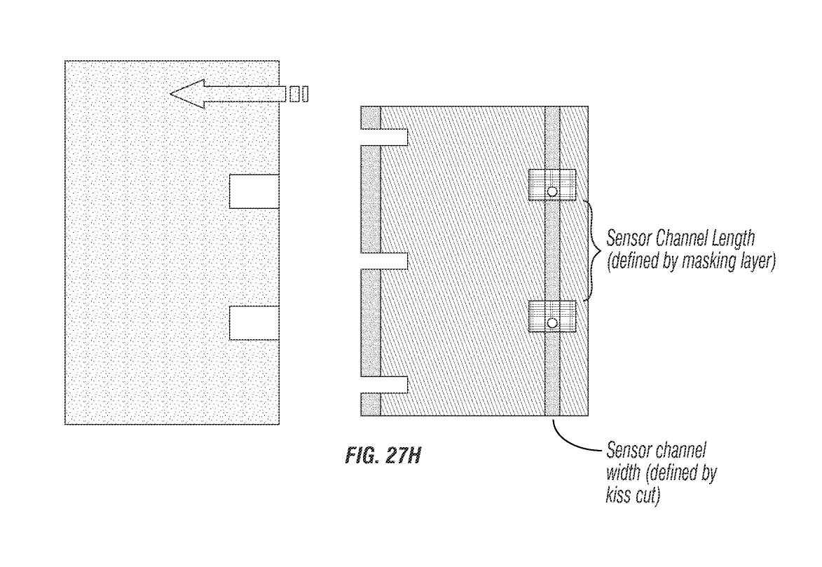

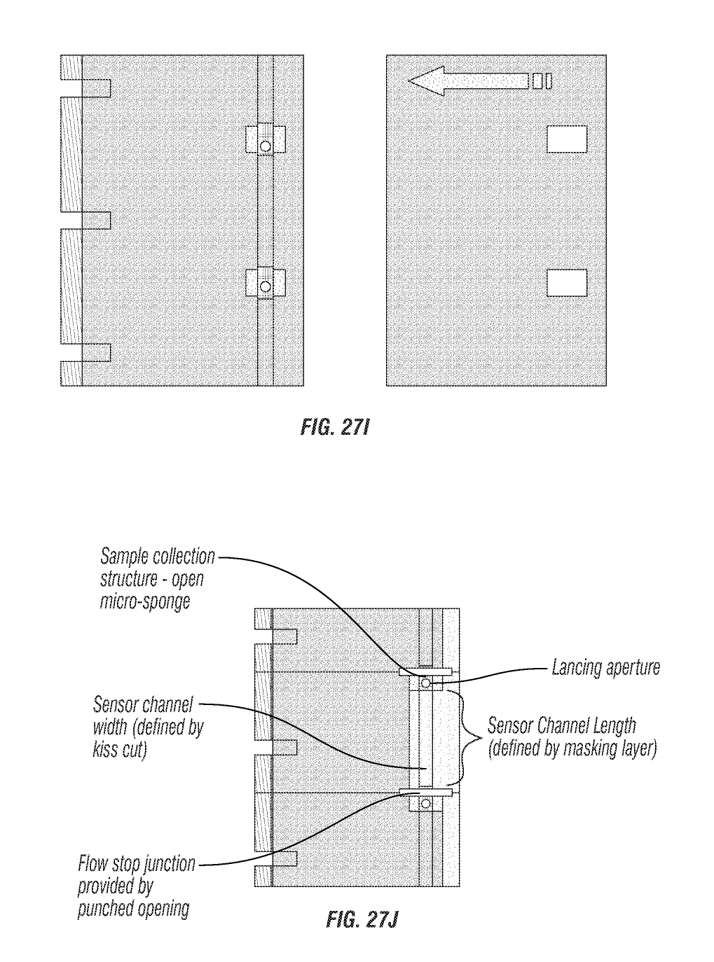

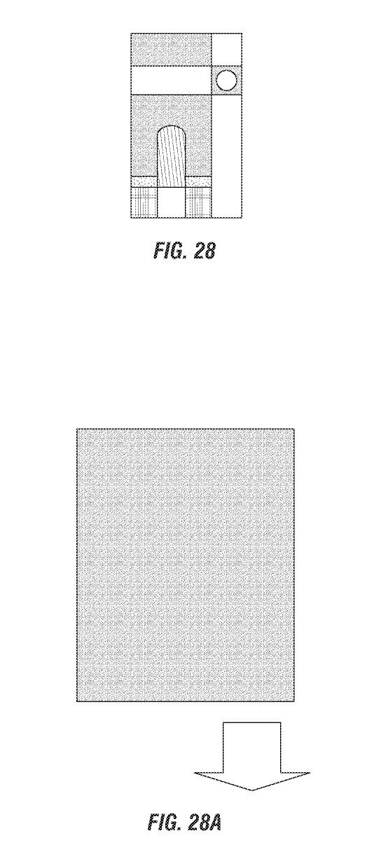

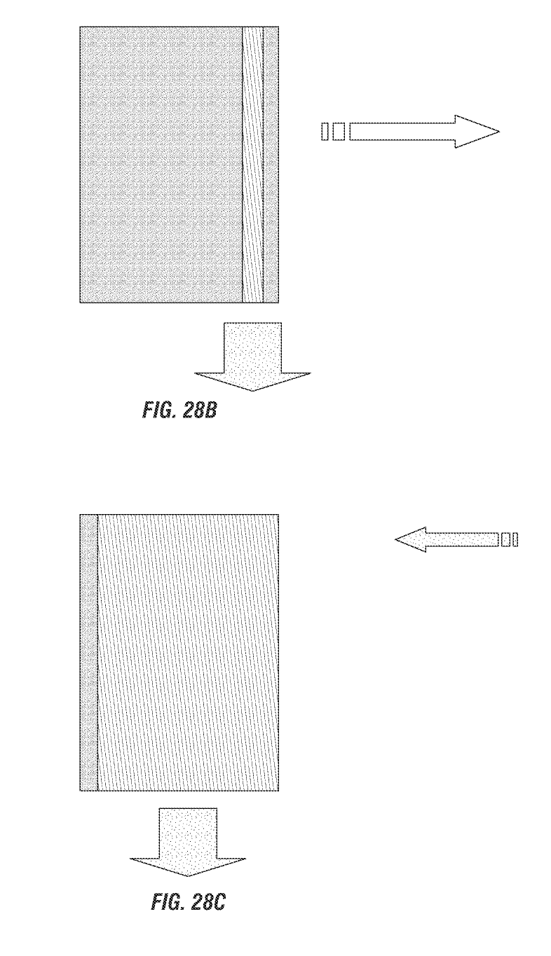

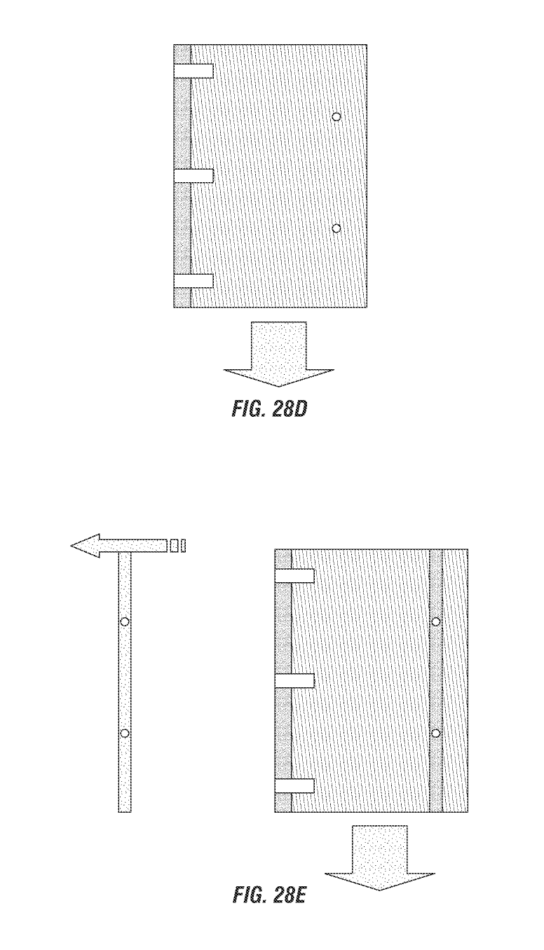

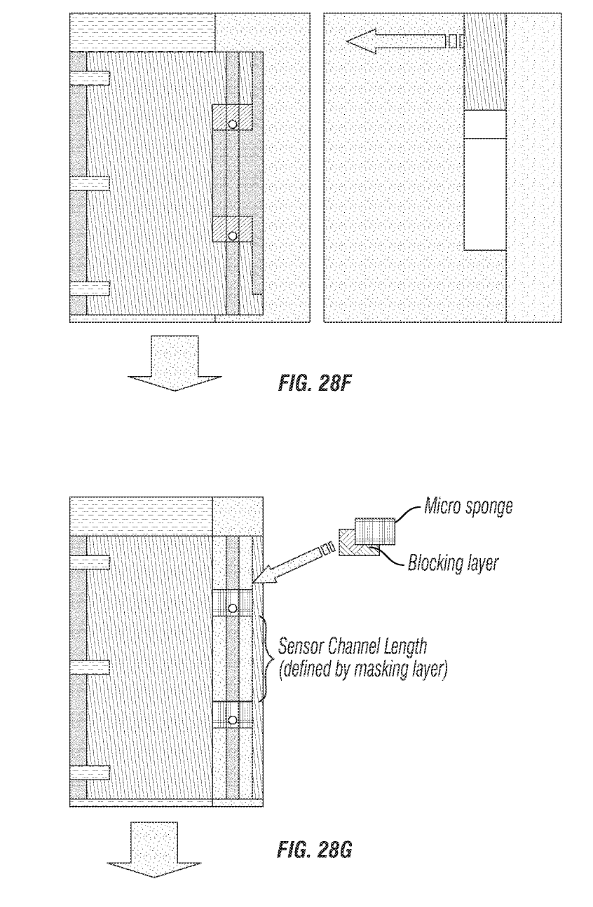

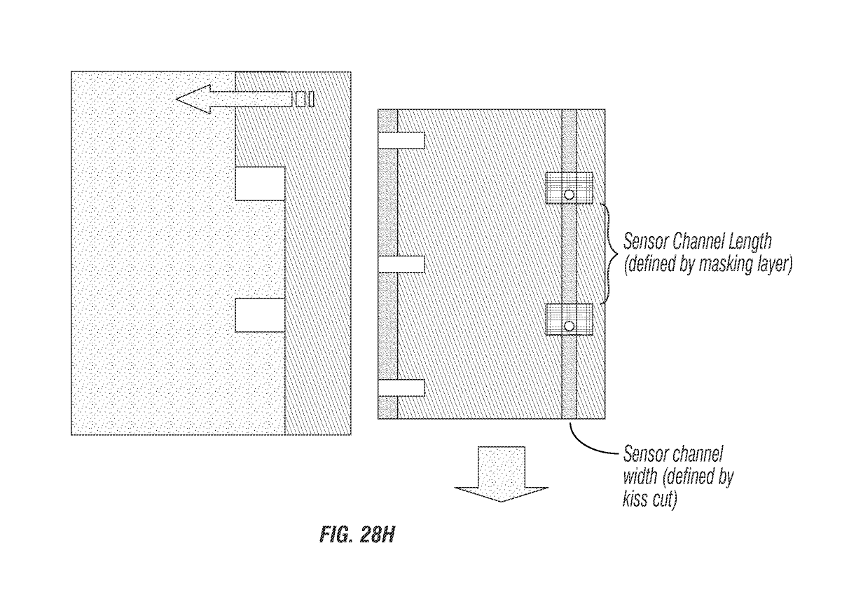

In another embodiment, illustrated in FIGS. 27 and 28, the sample capture is the end of the sensor/reaction zone 6108 channel. This embodiment maintains a separation of sample capture versus measurement.

FIG. 27 illustrates another embodiment of a strip 600 with sample capture. In this embodiment, a sample collection structure, with an aperture in a test strip substrate 6104 for lancing, an optional aperture in the test strip cover for blood collection and a micro-sponge material for collecting and transporting the blood within the collection structure is provided. A lancing aperture is provided in the substrate 6104 for a needle to pass through which may be about 1 mm. A sample collection aperture is optionally provided in the cover layer as an aperture for blood to access the sample collection micro-sponge structure. A blocking layer is located above the reagent layer, preventing reaction at other than the intended electrochemical cell. A micro-sponge layer is located above the blocking layer and within the sample collection and transport structures to facilitate sample collection and transport.

In this embodiment the sample collection/transport structure is at the end of the sensor/reaction zone 6108. Through a series of cutting, masking and deposition steps many different configurations of sensor/reaction zones 6108 can be created using the base structure.

In one method of making the FIG. 27 strip 600, the manufacturing process is as integral a part of the design of the test strip as the horizontal and vertical topology. The process flow for a strip 600 with sample capture is illustrated in FIGS. 27(a) through 27(i). In one method of manufacture, a roll of metal coated, palladium substrate 6104 material is the starting point for strip fabrication. The reactive reagent(s) for the analyte sensor, including but not limited to a glucose sensor, are deposited onto the metal coated substrate 6104 using, as a non-limiting example, slot, needle dispense or other methods. The substrate 6104 can be processed to have multiple reagent stripes for making multiple sensor/reaction zone 6108s in parallel.

The spacer layer 6106, with adhesives, is laminated onto the substrate 6104, covering the deposited reagent. The connector and penetrating member aperture features are punched onto the roll. The features locate the individual sensor/reaction zone 6108s on the roll. It is also possible to punch registration or alignment features at this step. The lancing aperture holes can also be punched, created, at a later step, thus preventing fouling of the hole by deposition steps. The sensor/reaction zone 6108 area is kiss cut into the spacer layer 6106. The spacer defining the sensor/reaction zone 6108 active area is removed at this time. A mask layer is aligned to the substrate 6104. The mask does not have significant critical alignment criteria, but roughly aligns to the lancing apertures. The masking is part of the printing of the blocking layer and can be applied separately or as part of the printing. The openings in the mask layer are printed, coated, with a blocking layer. The masking creates the structures for the sample capture area as well as defining the sensor/reaction zone 6108 channel length.

A micro-sponge layer is deposited in the sample capture/transport structures, on top of the blocking layer. The layer may be deposited via ink jet deposition, pad printing, roller printing or any other suitable method. The masking step may be conducted in conjunction with the printing step. The critical operation is to define the channel length with the masking layer. The mask is removed, exposing the sensor/reaction zone 6108 channel which is defined by the spacer layer 6106, width, and the mask/micro sponge layers, length. A metalized cover layer is laminated onto the test strip structure. This is applied as a conversion step from rolled materials. The gold layer has pre-punched openings. The registration requirements are only to roughly align the openings to the micro sponge.

When the release liner is removed from the spacer to expose the adhesive, the micro-sponge and blocking layer is then left only in the channel. Alternately, this layer can be pre-punched with the sample capture aperture. In this case, alignment will be more critical. The assembled roll of test strips 600 are singulated into individual, ribbons or blocks of completed sensor/reaction zones 6108s for subsequent processing. If necessary, the step can use a die punching operation to precisely define the glucose sensor/reaction zone 6108 channel. The lancing aperture in this step can be punched, instead of earlier, to facilitate keeping the hole from being fouled by chemistry such as block and sponge.

In the embodiment illustrated in FIG. 28, sample capture is provided through the top of sensor/reaction zone 6108. In this embodiment, sample capture is presented through a cover feature, directly on the sensor/reaction zone 6108. This is a simple approach with direct fluidic connection between sample capture and the sensor/reaction zone 6108 but does not lend itself to separation of function.

The test strips of the FIG. 28 embodiment can be made with the process steps of FIGS. 28(a) through 28(j). From a roll, a palladium coated substrate 6104 has a surface treatment. A slot, or other method, is coated to add reagent chemistry including but not limited to GDH-FAD w/ mediator. A roll based spacer layer 6106 is laminated. The substrate 6104 and adhesive spacer are punched to feature contact legs and a penetrating member aperture. Optionally, a feature for registration of subsequent steps can be included. The spacer layer 6106 is kiss cut, creating a sensor/reaction zone 6108 area, and spacer area and a defined sample capture structure is removed. Registration of cut to penetrating member apertures maybe required. The spacer layer 6106 covering the sensor/reaction zone 6108 is removed. A mask layer is aligned to the substrate 6104. The mask does not have significant critical alignment criteria, but roughly aligns to the lancing apertures. The masking is part of the printing of the blocking layer and can be applied separately or as part of the printing Openings in the mask layer are printed, such as by coating, with a blocking layer and micro sponge. The masking creates the structures for the sample capture area as well as defining the sensor/reaction zone 6108 channel length The mask is then removed, exposing the sensor/reaction zone 6108 channel which is defined by the spacer layer 6106 width and the mask/micro sponge layers, length. The gold layer is laminated.

This is applied as a conversion step from rolled materials. The gold layer has pre-punched openings. The registration requirements are only to roughly align the openings to the micro sponge. When the release liner is removed from the spacer to expose the adhesive, the micro-sponge and blocking layer is then left only in the channel A covered sample capture structure can be achieved by pre-punching the gold layer appropriate and doing an aligned lamination. The strips are then punched and cut. Optionally, it is possible to punch the lancing aperture in this step, instead of earlier to facilitate keeping the hole from being fouled by chemistry, block and sponge.

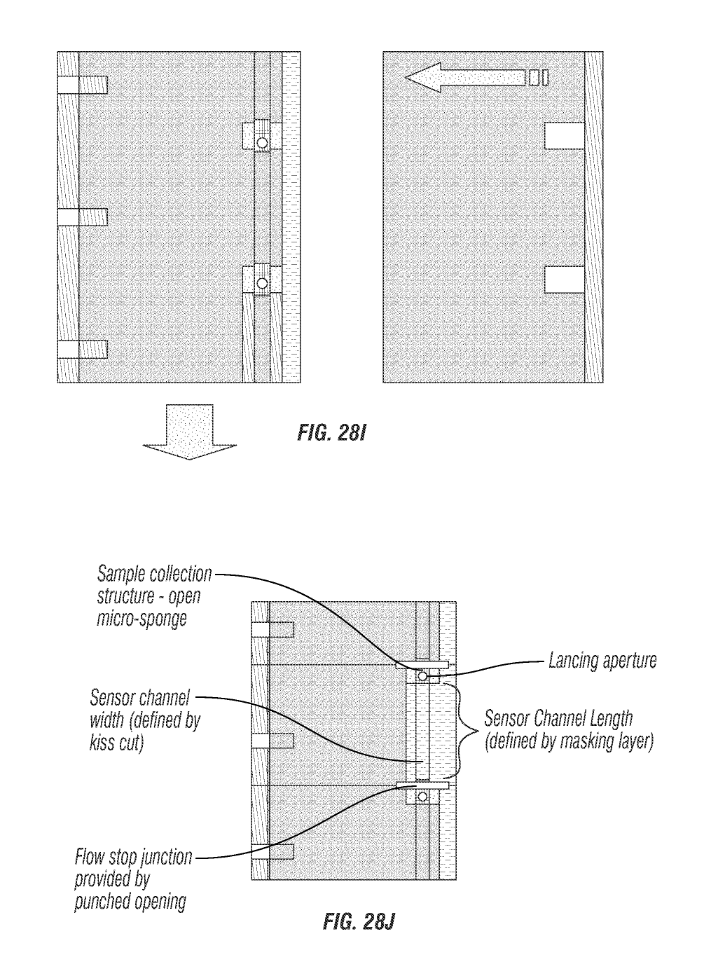

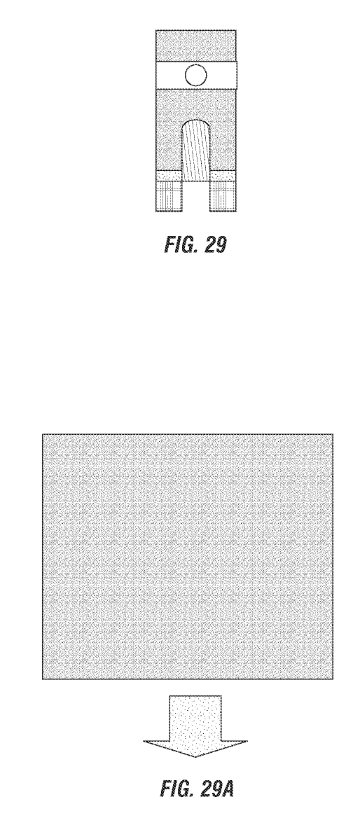

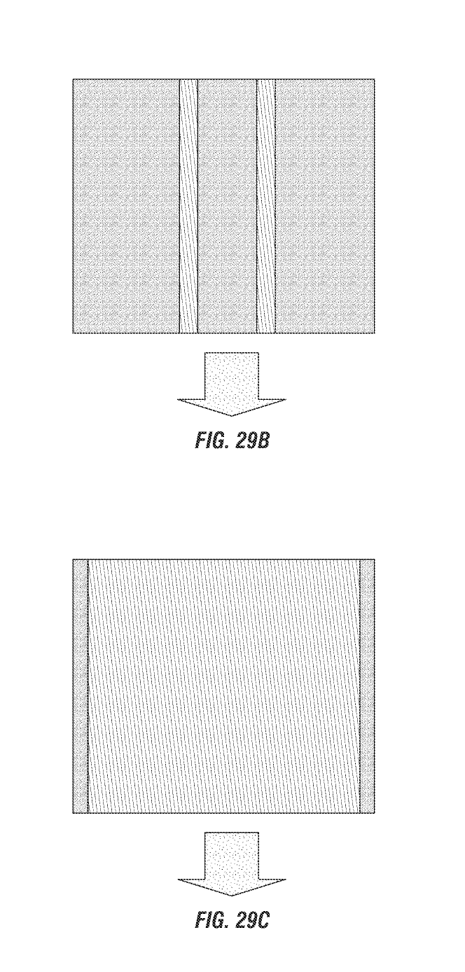

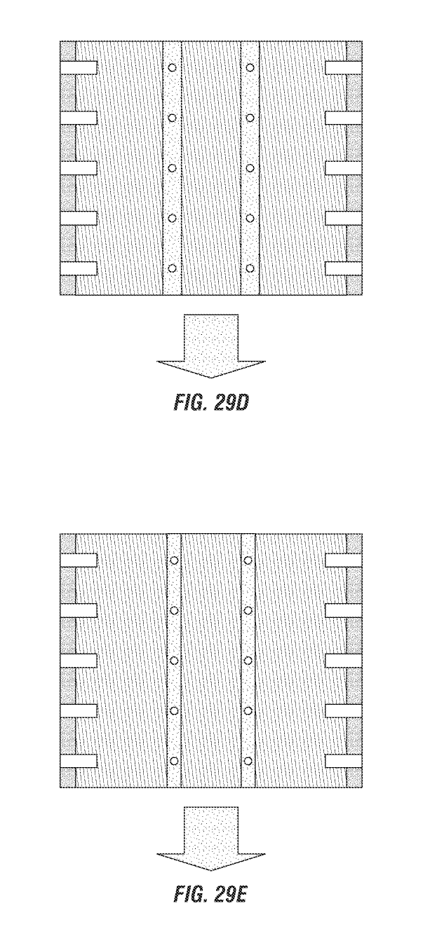

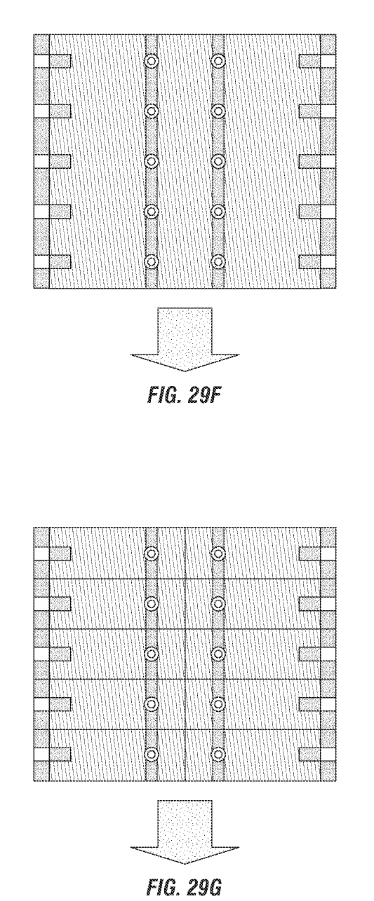

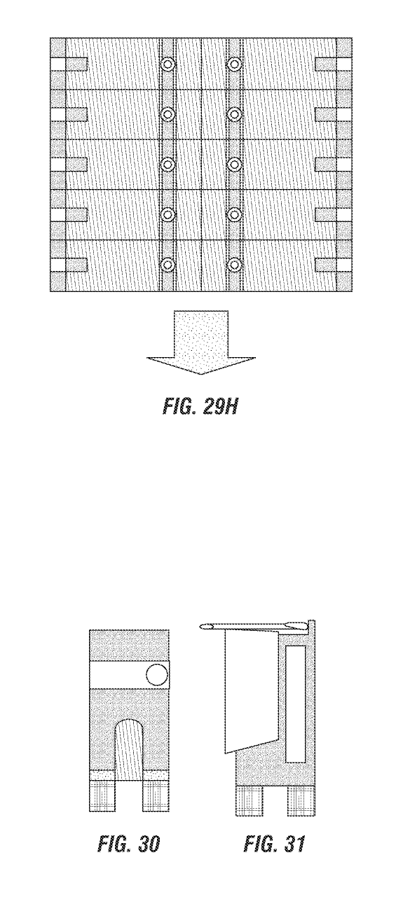

In the embodiments illustrated in FIGS. 29 and 30, sample capture is placed on the edge of the sensor/reaction zone 6108 channel, and impinges into the sensor/reaction zone 6108. This provides direct fluidic connection between the sample capture and the sensor/reaction zone 6108.

In the FIG. 29 embodiment, a lancing aperture is provided in the substrate 6104 for a needle to pass through, which as a non-limiting example can be about 1 mm. A sample collection aperture is optionally provided in the cover layer as an aperture for blood to access the sample collection micro-sponge structure. A micro-sponge layer is optionally provided. In the FIG. 29 embodiment, the sample collection/transport structure is at the center of the sensor/reaction zone 6108, and as shown, is within the cell. Through a series of cutting, masking and deposition steps, different configurations of sensor/reaction zones 6108 using the base structure can be created. Examples of other configurations include but are not limited to: a sensor/reaction zones 6108 with off-center through hole; a sensor/reaction zones 6108 with micro sponge in the channel; a sensor/reaction zones 6108 with sample capture structure in the channel and the like.

The strips 600 illustrated in FIGS. 29 and 30 can be manufactured with the following steps illustrated in FIGS. 29(a) through 29(h). A palladium coated substrate 6104 is provided with a surface treatment on a roll. A slot, or other suitable method, is coated to add reagent chemistry including but not limited to GDH-FAD w/ mediator. A spacer layer 6106, on a roll, is laminated. The substrate 6104 and adhesive spacer are punched to feature contact legs and penetrating member aperture, located in the sensor/reaction zones 6108 area. If required, a feature for registration of subsequent steps can be added.

The spacer layer 6106 is kiss cut and a spacer area defining the sensor/reaction zones 6108 structure are removed. A gold cover layer is applied, which requires registration required. A roll cut is performed to singulate the sensor/reaction zones 6108 as a single, ribbon, block and the like. Optionally, a micro sponge is in the channel on the gold cover film. The gold cover film is pre-punched for sample capture aperture and coated, on its underside, with micro sponge to enhance fluidic flow.

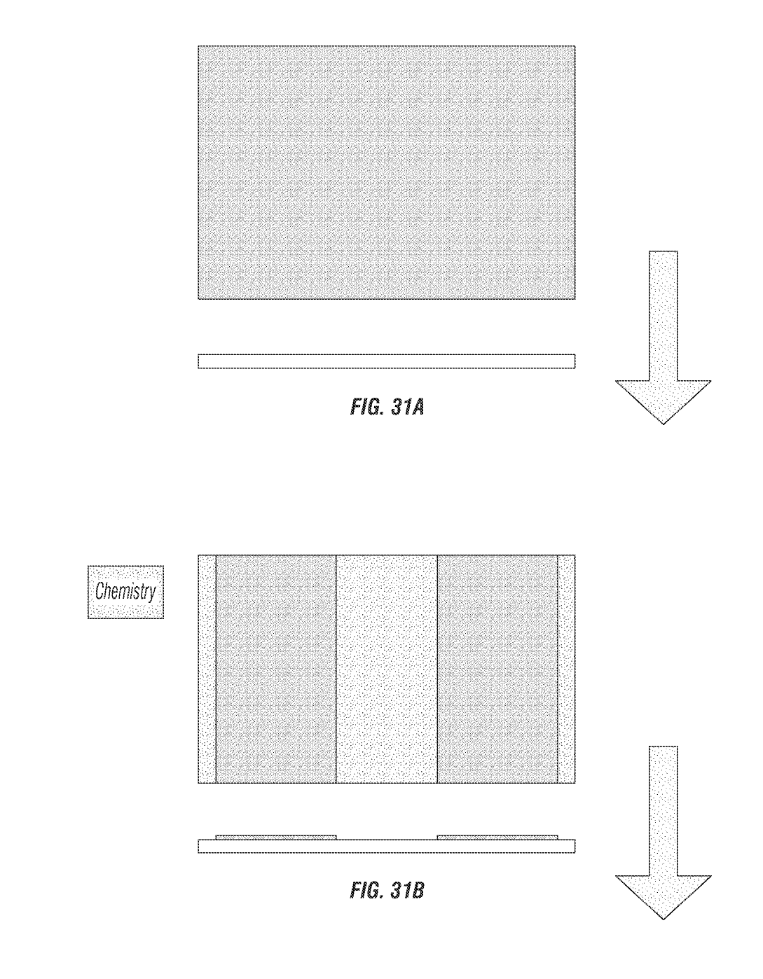

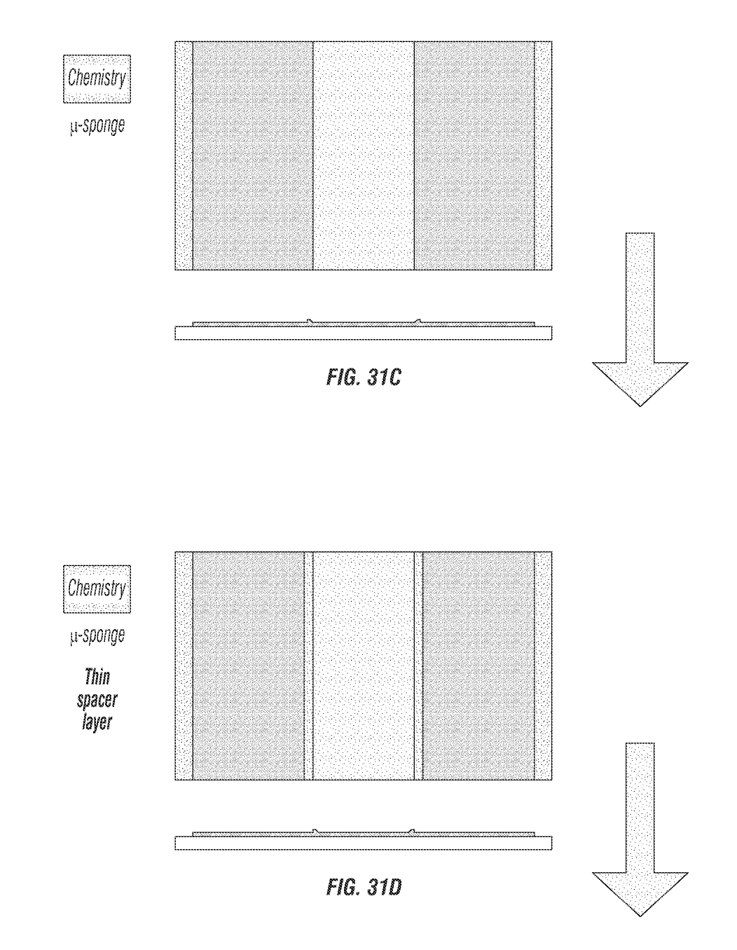

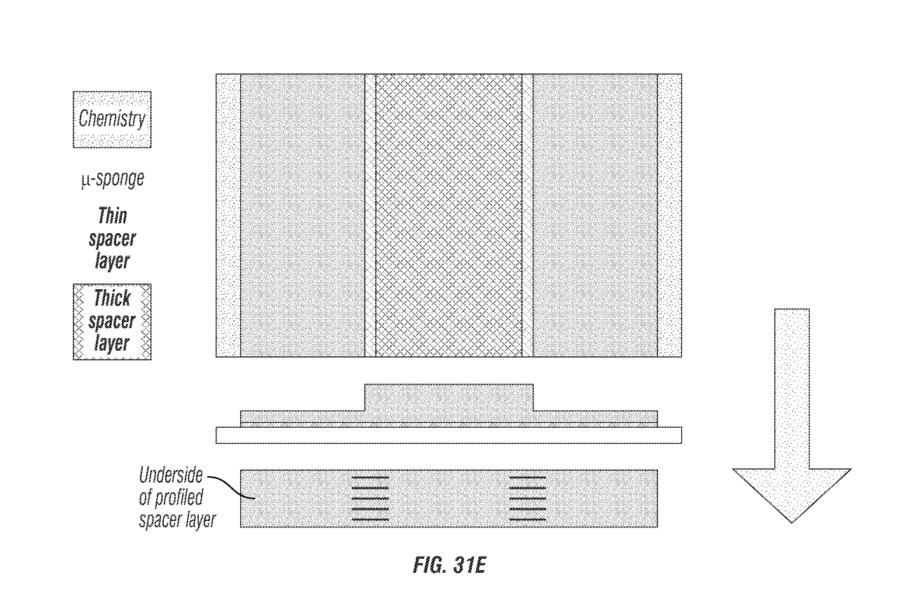

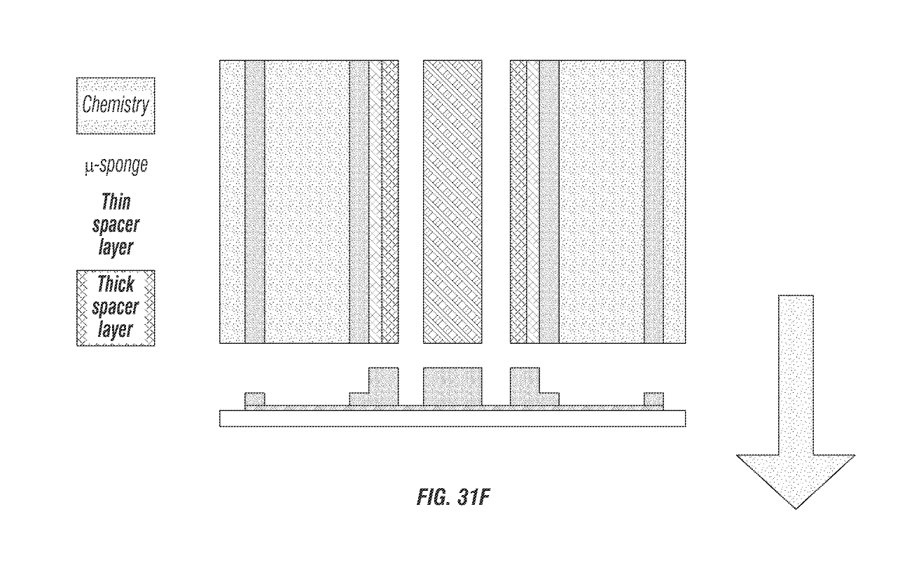

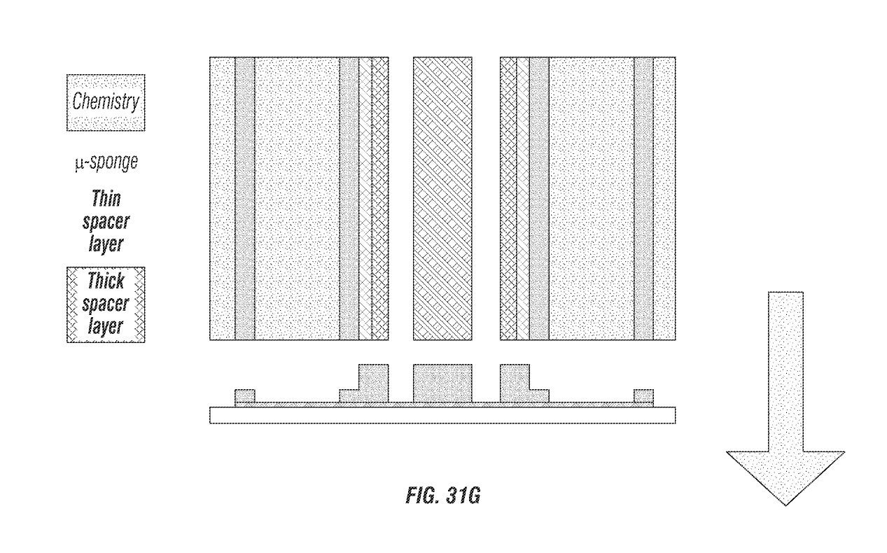

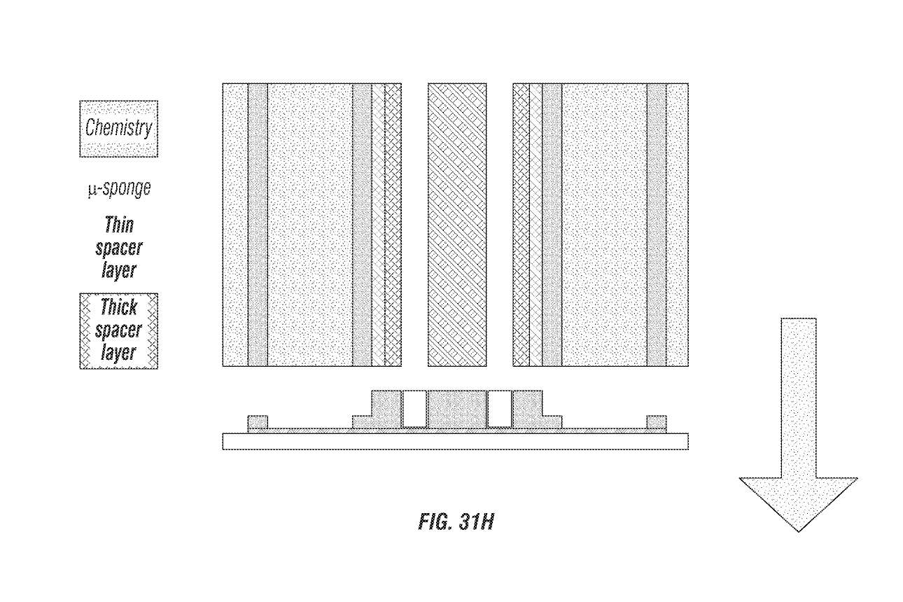

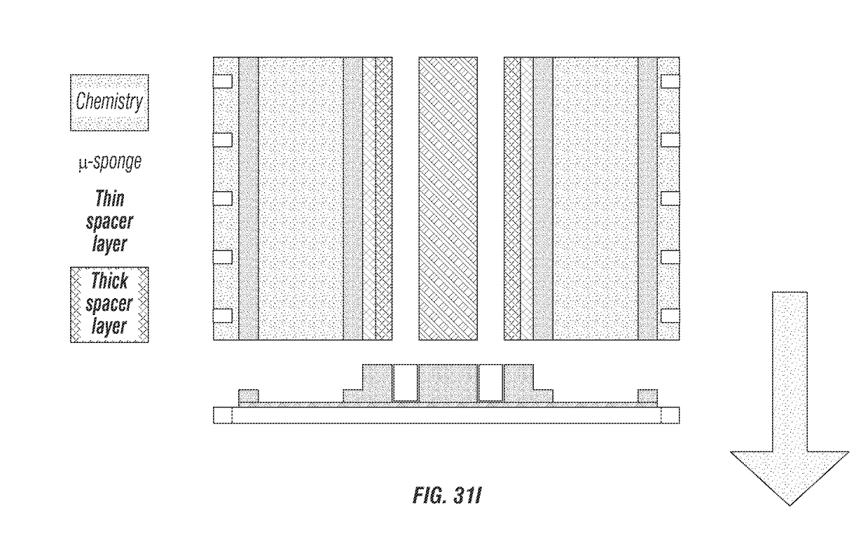

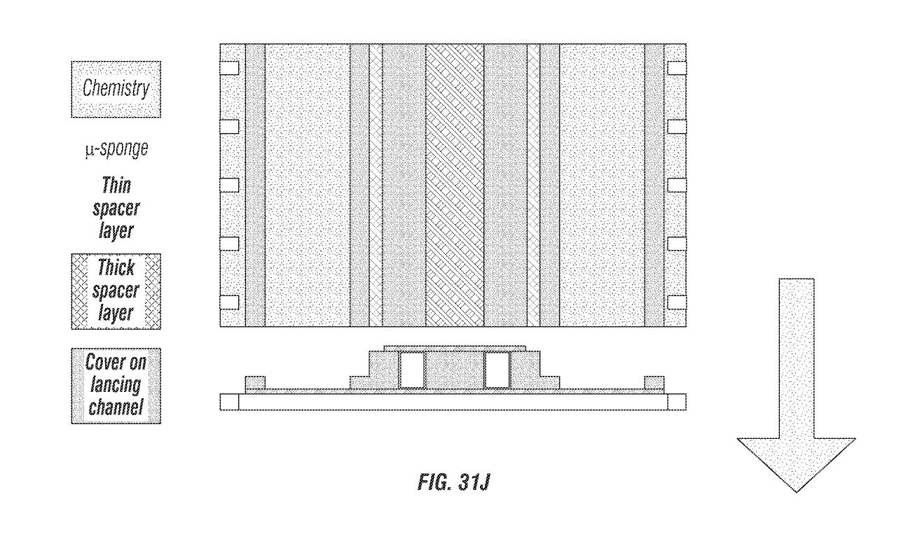

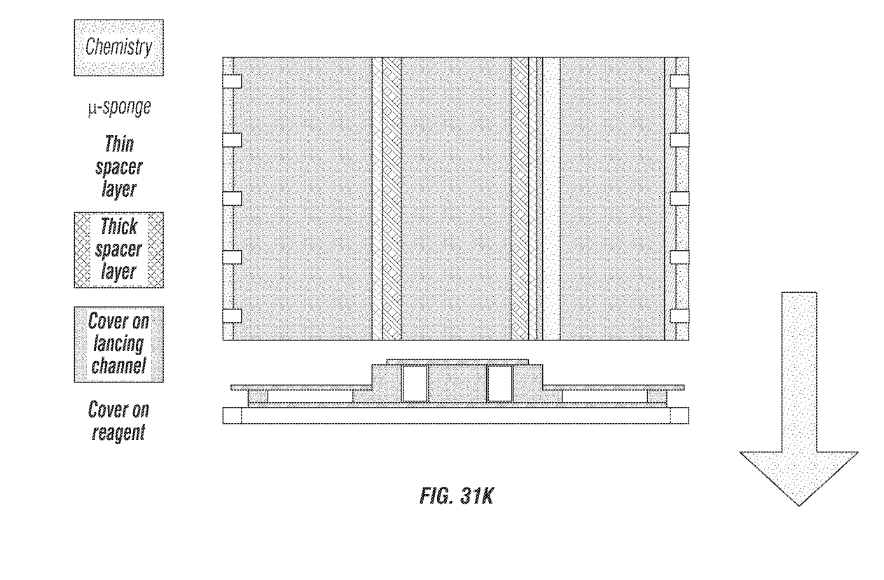

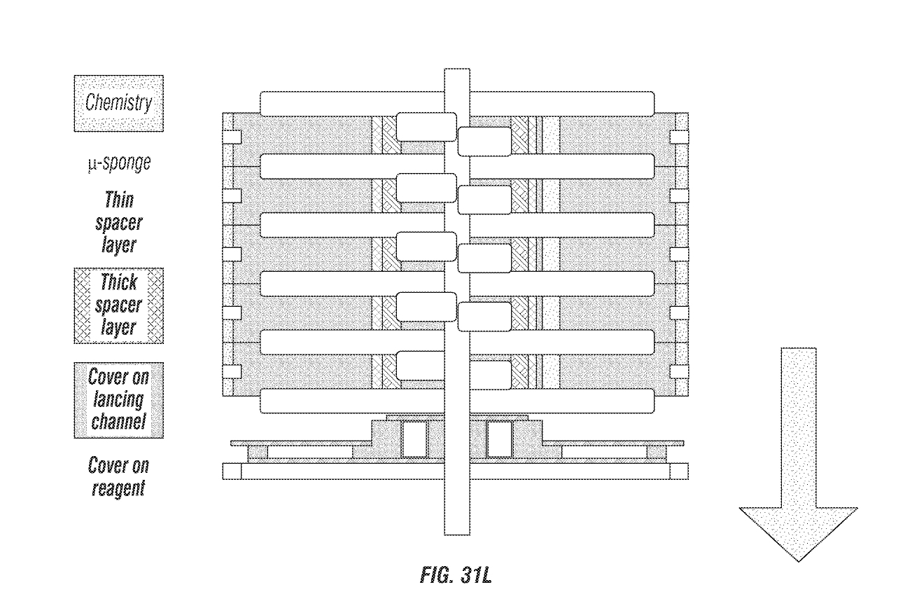

FIG. 31 illustrates an embodiment of a strip 600 with a sample capture structure orthogonal to a plane of the strip. A micro-sponge can surround the penetrating member channel and connects to the reaction cell.

The FIG. 31 embodiment can be made with a palladium coated substrate 6104 on a roll that is slot coated to add reagent chemistry, including but not limited to GDH-FAD w/ mediator, and the like, as illustrated in FIGS. 31(a) through (l) It is slot coated to add a micro-sponge. In one embodiment, the micro-sponge can be the cover reagent. Adhesive layers are added on the edges to the spacer layer 6106. A profiled adhesive spacer layer 6106 is also added in the middle. The spacer layer 6106 has grooves to connect a center channel to the chemistry. Three separate spacer layers 6101 can be used. The spacer layers 6106 are kiss cut and the waste is then removed. This defines the reagent area and the lancing channel.

The lancing channel is filled with micro-sponge which is the rabbet out to form a U-shaped groove in the lancing channel. Contact legs are defined by punching. A cover is laminated for the lancing channel. Gold is then laminated on to cover the reagent. The cover has micro-sponge on an underside that is about the width of lancing channel. At this point, the lancing channel surrounds the penetrating member with micro-sponge. Roll punch can be used to singulate the strips 600.

In another embodiment, a wicking plug is used in the sample capture feature, which can be for a through cover configuration. A hydrophilic wicking plug can be employed that passes through the cover of the channel. This embodiment is a variant of the through the top but adds a fluidic member to collect sample and to move fluid through the opening.

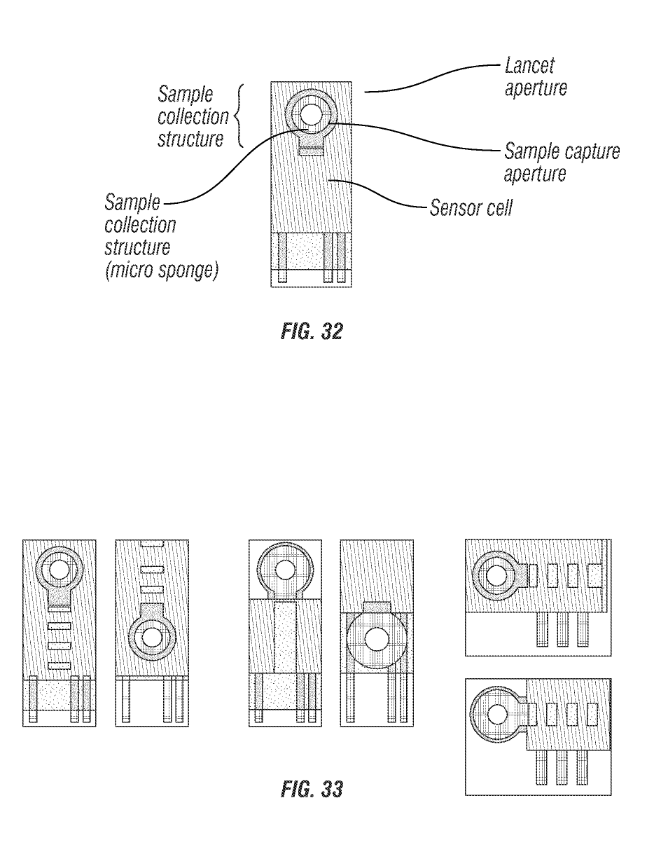

In another embodiment of the present invention, illustrated in FIG. 32, the analyte sensor of the present invention includes test strip 600 that integrates the following structure and capabilities in an effective way to, (i) to generate a sample is through using a controlled lancing event, where the profile of the lancing event is controlled; (ii) collect a blood sample and have the lancing event occur such that a lancing needle path is perpendicular to the plane of a circular sample collection structure; and (iii) transport the sample, once collected, through a hydrophilic treated capillary connecting the sample collection to the sensor.

In this embodiment, the sample capture structure includes an aperture in a test strip 600 substrate 6104 for lancing.

Optionally, an aperture in a test strip cover is provided for blood collection along with a micro-sponge material for collecting and transporting the blood within the collection structure. In this embodiment, a lancing aperture is provided in the substrate 6104 for a penetrating member to pass through. In one embodiment, the lancing aperture is about 1 mm. A sample capture aperture is optionally provided in the cover layer, as an aperture for blood to access the sample collection micro-sponge structure. A sample collection structure, in this case a micro-sponge layer, is optionally located within the sensor structure to facilitate sample collection and transport

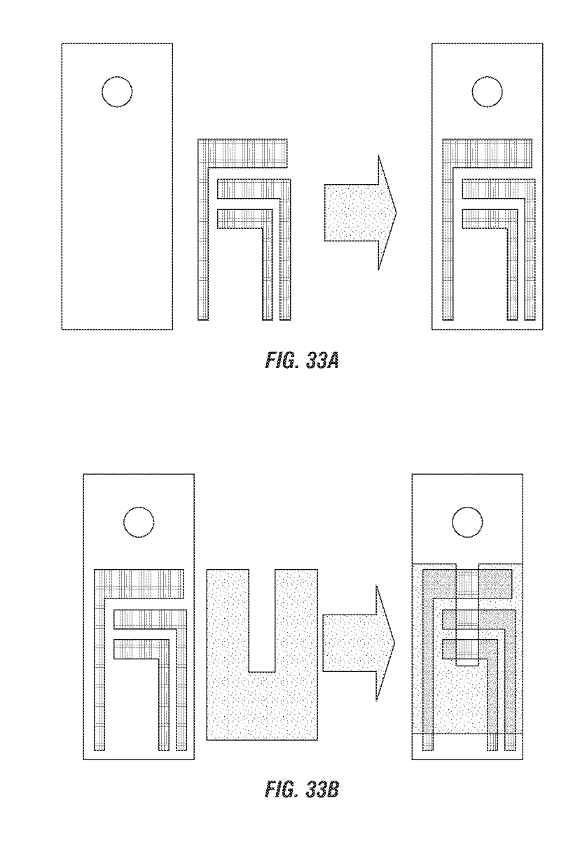

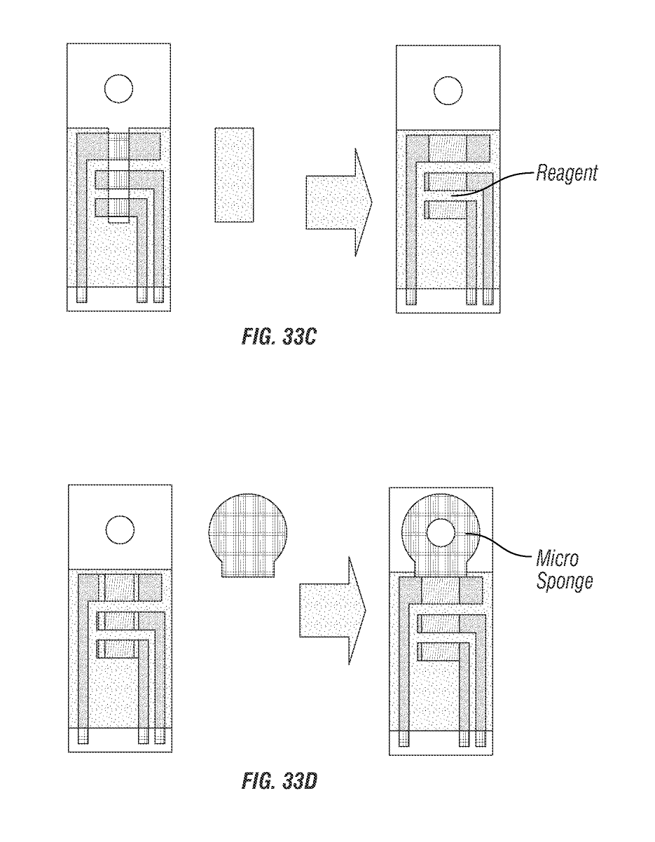

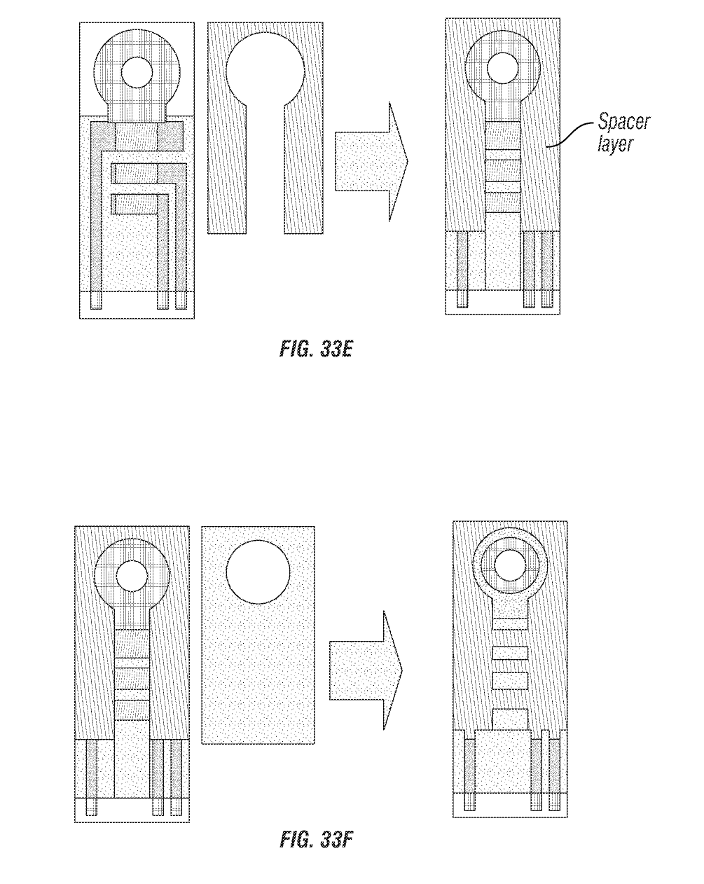

In the FIG. 32 embodiment, an integrated sensor, the sample collection/transport structure, is at the end of the sensor cell, and, as shown, is located at the end of the test strip 600. Through a series of cutting, masking and deposition steps, a variety of different configurations can be provided using the base structure, as illustrated in FIG. 33.

In one embodiment of manufacturing the strip 600 of FIGS. 32 and 33, a conductive layer is screen printed onto the strip substrate 6104, which can be plastic as described above. In this case, the conductive layer can be a carbon ink. The registration is made to the lancing aperture, loose which is pre-punched into the substrate 6104 as illustrated in FIG. 33(a).

As shown in FIG. 33(b), an insulation layer is printed onto the step 1 output. As a non-limiting example, Ercon E6110-116 Jet Black Insulator Ink can be used. The registration is made to the carbon pads, loose. The Insulation layer forms the width of the electrodes.

Referring now to FIG. 33(c) reagent is printed onto the step 2 output. The reagent can be, as a non-limiting example, glucose oxidase, a co-enzyme, a mediator, and a hydrophilic filler material is used. The reagent layer provides the chemistry for the assay as well as a hydrophilic layer to promote the filling of the sensor cell. The registration is made to the carbon pads, loose.

The micro sponge is printed onto the step 3 output. The registration is made to the lancing aperture, loose, as shown in FIG. 33(d).

As illustrated in FIG. 33(e), the spacer is screen printed onto the step 4 output. As a non-limiting example, the spacer can be an acrylic copolymer pressure sensitive adhesive (e.g., available from Tape Specialties, Ltd., Tring Herts, United Kingdom). The registration is made to the lancing aperture, loose. The spacer forms the sensor channel width and thickness, both of which are important for the performance of the sensor.

The cover slip is laminated onto the adhesive spacer layer, FIG. 33(f). As a non-limiting example, the cover slip can be a polyester sheet, treated to have a hydrophilic surface, facing the sensor cell, and optically transparent to facilitate user recognition of the cell filling. The registration is made to the lancing aperture which is fairly tight. The sample capture structure is formed and is fluidicly tightly coupled to the sensor cell.

In one embodiment, a protective cover, such as paper, is on the cover layer as a mask, an ink jet is sprayed as a hydrophilic layer (e.g., a membrane or micro-sponge) onto the sample capture structure after cover lamination. The mask results in a closely fluidic integrated, hydrophilic sample capture structure.

In another embodiment of the present invention strip 600 incorporates a lancing hole or indentation on the edge. This is a sample-capture feature configured to maximize the likelihood of capturing a sample of blood immediately following lancing, and a sample-collection feature which provides a favored path for the blood to enter the test strip. Further, the lancing, sample capture, sample collection and sample transport features can be monitored such that a proper and/or improper sample delivery to a biological sensor can be determined.

This embodiment includes the combination of lancing, sampling, and measuring a blood analyte. This embodiment includes: an aperture for a penetrating member; sample capture feature; sample collection feature; sample transport feature and a sample detection feature. The sample transport pathway moves a biological fluid to a specified portion of the strip 600 for reaction with a reagent and measure of the reaction products.

The sample-capture feature can be shaped in a non-planar way to maximize the ratio of the area of the sample-capture feature to the area of the skin surrounded by the sample-capture feature.

The strip 600 can be fabricated such that the penetrating member path is provided by an indentation in one edge of the test strip and in which the sample-collection and sample-capture features substantially surround the indentation.

The sample-collection feature can include a micro-fluidic micro-sponge that is hydrophilic for the analyte and substantially surrounds the penetrating member wound in close proximity to the wound. Again, close proximity can be ≦300 μm from the skin which includes touching the skin, the micro-sponge forms an annular micro-fluidic capillary layer, and a hydrophobic area to prevent unwanted wetting by the analyte.

As a non-limiting example, the sample-collection feature can capture a sample of analyte between 100 nano liter and 5,000 nano liter.

The transport pathway can be a micro-fluidic channel from the sample-collection and sample-capture features to a specified portion of the test strip. A volume of the transport pathway can be <10% of the total volume of the test strip.

The body fluid sample of the analyte is obtained either by (i) lancing through the path for the penetrating member and filling the sample-capture structure with analyte while the sample-capture structure is in close proximity to the skin; or (ii) lancing a skin surface such as the finger and an expressed sample of analyte is manually placed on to the sample-capture structure.

In another embodiment, the transport pathway can created by covering the substrate 6104 of the test strip with a cover layer which provides a two-dimensional capillary area over which the analyte spreads automatically by means of capillary forces and in which reagent exists within the capillary area. The optical properties of the two-dimensional capillary area are changed in proportion to the concentration of the analyte and measurement of the concentration is by optical reflectance, transmission, or fluorescence.

In another embodiment, the sample-collection feature is a micro-fluidic hydrophilic structure, including but not limited to a micro-sponge, membrane, film, and the like, containing reagent which reacts with the analyte. The products of the reaction are measured optically or electrically by voltage, charge, current and the like.

As a non-limiting example, the sample capture feature can be an aperture providing a penetrating member path, a structure which substantially surrounds the penetrating member wound in close proximity to the wound. Close proximity can be ≦300 μm from the skin which includes touching the skin, and a hydrophobic area to prevent unwanted wetting by the analyte. In one embodiment, the detection mechanism is integrated into one or more of the sample collection, sample capture and sample transport features to detect the proper and/or improper supplying of sample to the sensor. The detection mechanism can be electrical including but not limited to, conductive, capacitive, resistive, inductive, and the like. The measured reaction can be an electrochemical measured as voltage, charge, or current.

In one embodiment, the detection mechanism is optical such as, transmission, reflective, emitting from excitation, and the like, used in any wavelength or combination of wavelengths from infrared, 2000 nm through ultraviolet 400 nm. The reaction with the reagent is such that the optical properties of the specified portion of the strip 600 change during the reaction and the measurement of the reaction is by optical reflection, optical transmission, or optical fluorescence.

The specified portion of the strip 600 is a volume above a set of planar electrodes, or the volume between a set of opposed electrodes 624, 626, which can be 2, 3, or 4 electrodes. The ratio of the area of the electrodes to the volume of the analyte is not affected by the volume of analyte in the sample-collection feature.

FIG. 34 is a cross section of the strip 600 and illustrates the (i) penetrating member path through the strip 600; (ii) sample capture feature with cover that has hole larger than the micro sponge with a hydrophobic on the upper surface; (iii) sample collection feature: the hydrophilic micro sponge surrounding the penetrating member and exposed to the skin on a finger when in close proximity; and spacer forms the walls of the sample transport feature.

FIG. 35 is an exploded view of the FIG. 34 embodiment.

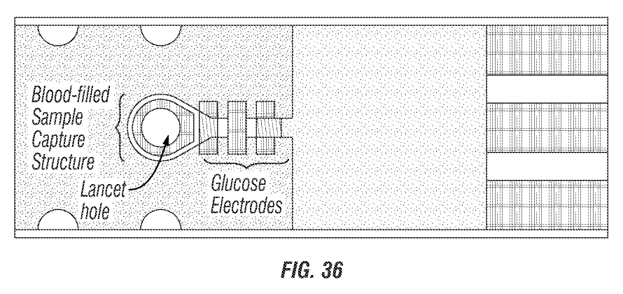

FIG. 36 another drawing of the strip 600.

The publications discussed or cited herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. All publications, patents, and patent applications mentioned herein are incorporated herein by reference to disclose and describe the structures and/or methods in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A test strip device, comprising:
a first substrate with a first electrode;
a second substrate with a second electrode, with a fluid passage way between the first and second substrates;
a spacer layer that includes an aperture coupled to the fluid passage way and positioned between the first and second electrodes;
a reaction zone/sensor formed between the first and second electrodes; and
a hydrophilic sample collection structure.

2. The strip device of claim 1, wherein the sample collection structure includes, at least one of, a micro sponge, a hydrophilic layer, an annular capillary surrounding the lancing needle/wound; and a hydrophobic coating on the outside facing surface of the cover film, with the sample capture structure surrounds the lancing wound site.

3. A test strip device for testing a biologic analyte obtained by lancing a finger, comprising:
an aperture in the test strip providing a path for a penetrating member;
a sample-capture feature;
a sample-collection feature, and a transport pathway to move the analyte to a specified portion of the test strip for reaction with a reagent and measurement of the reaction products.

4. The device of claim 3, wherein the sample-capture feature includes an aperture providing a penetrating member path, a structure which substantially surrounds the penetrating member wound in close proximity to the wound, and a hydrophobic area to prevent unwanted wetting by the analyte.

5. The device of claim 4, wherein the sample-capture feature is shaped in a non-planar way to maximize the ratio of the area of the sample-capture feature to the area of the skin surrounded by the sample-capture feature.

6. The device of claim 3, wherein the sample-collection feature includes a micro-fluidic micro-sponge which is hydrophilic for the analyte and substantially surrounds the penetrating member wound in close proximity to the wound, and a hydrophobic area to prevent unwanted wetting by the analyte.

7. The device of claim 6, wherein the sample-collection feature can capture a sample of analyte between 100 nano liters and 5,000 nano liters.

8. The device of claim 3, wherein the transport pathway includes a micro-fluidic channel from the sample-collection and sample-capture features to a specified portion of the strip.

9. The device of claim 3, further comprising:
a detection mechanism is integrated into one or more of the sample collection, sample capture and sample transport features to detect the proper and/or improper supplying of sample to the test strip in which the sample-collection and sample-capture features substantially surround said indentation.

10. A test strip device, comprising:
an aperture in the test strip providing a path for a penetrating member;
a sample-capture feature;
a sample-collection feature; and
a transport pathway created by covering the substrate of the test strip with a cover layer which provides a two-dimensional capillary area over which the analyte spreads automatically by means of capillary forces and in which reagent exists within said capillary area which reacts with the analyte such that the optical properties of the two-dimensional capillary area are changed in proportion to the concentration of the analyte and measurement of said concentration is by optical reflectance, transmission, or fluorescence.

11. A test strip device, comprising:
an aperture in the test strip providing a path for the penetrating member;
a sample-capture feature; and
a sample-collection feature in which the sample-collection feature is at least one of, a micro-fluidic hydrophilic structure containing reagent which reacts with an analyte.

12. The device of claim 11, wherein products of the reaction are measured optically.

13. The device of claim 11, wherein products of the reaction are measured electrically by at least one of, voltage, charge, and current.
